(19) United States
(12) Patent Application Publication
(10) Pub. No.: US 2023/0149428 A1
(43) Pub. Date: May 18, 2023

(54) METHOD OF TREATING GENE THERAPY ASSOCIATED TOXICITY WITH ANTIBIOTICS

(71) Applicant: Asklepios BioPharmaceutical, Inc., Research Triangle Park, NC (US)

(72) Inventors: Sam HOPKINS, Research Triangle Park, NC (US); Leigh Ervin, Research Triangle Park, NC (US)

(73) Assignee: Asklepios BioPharmaceutical, Inc., Research Triangle Park, NC (US)

(21) Appl. No.: 17/797,633

(22) PCT Filed: Feb. 12, 2021
(86) PCT No.: PCT/US21/17818

§ 371 Date: Aug. 4, 2022

Related U.S. Application Data
(60) Provisional application No. 62/976,755, filed on Feb. 14, 2020.

(51) Int. Cl. A61K 31/65; C12N 15/86; C12N 9/24; A61K 38/47; A61P 39/00; A61K 31/573

(52) U.S. Cl. CPC A61K 31/65; C12N 15/86; C12N 9/2402; C12Y 302/0102; A61K 38/47; A61P 39/00; A61K 31/573; C12N 2750/14143; C12N 2750/14171

(57) ABSTRACT

Disclosed herein are methods of treating a toxicity in a subject receiving recombinant viral vector, such as a recombinant adeno-associated viral AAV (rAAV) vector comprising co-administration of an antibiotic and a viral vector. In one embodiment, the antibiotic is a tetracycline or macrolide family member. Also provided herein are compositions comprising an antibiotic and a viral vector.

### CROSS-REFERENCE PARAGRAPH

This invention is a 35 U.S.C. § 371 National Phase Entry Application of International Patent Application No. PCT/US2021/017818 filed on Feb. 12, 2021, which designated the U.S., which claims benefit under 35 U.S.C. § 119(e) of U.S. Provisional Application 62/976,755 filed on Feb. 14, 2020, the contents of which are incorporated herein in their entireties by reference.

### SEQUENCE LISTING

The instant application contains a Sequence Listing which has been submitted electronically in ASCII format and is hereby incorporated by reference in its entirety. Said ASCII copy, created on Mar. 17, 2021, is named 046192-096800WOPT_SL.txt and is 4,297 bytes in size.

### FIELD OF THE INVENTION

The present invention relates to the field of gene therapy, specifically treating adverse side effects, e.g., toxicity, associated with high therapeutic dose gene therapy.

### BACKGROUND OF THE INVENTION

Viral vectors, such as recombinant adeno-associated viral vectors (rAAV vectors), are being used as gene delivery vehicles for a wide range of transgenes in pre-clinical and clinical studies for many pathological indications. Delivery of large amounts of viral vectors encoding a therapeutic transgene is frequently advantageous as it can result in high expression levels of the transgene. However, this level of large administration can sometimes lead to complications such as liver-toxicity and induction of an inflammatory response. It is therefore desirable to achieve high levels of transgene expression without the toxicity and inflammation that can accompany administration of large amounts of viral vectors.

Delivery of small amounts of viral vectors, including recombinant adenoviral vectors, encoding a therapeutic transgene can lead to low or undetectable expression levels of the transgene, thus may be undesirable. In contrast, small increases in the amount of viral vectors administered to a subject can lead to disproportionately large, e.g., non-linear increases in expression levels of the transgene encoded by the viral vector (Tao et al, Molecular Therapy 3: 28-35 (2001), involving the human IFN-β transgene).

Another complication associated with delivery of large amounts of viral vectors in gene therapy is the generation of a host inflammatory response, potentially resulting in clinical toxicity and impairment of gene transfer efficacy. Acute inflammatory responses have been observed, for example, in many animal models after a high-dose administration of adenoviral vectors, and vigorous cytokine release is likely the cause of acute toxic reactions seen in some human trials (Crystal et al, Nature Genetics 5:42-51 (1994); McElvaney et al, Nature Medicine 1: 182-184 (1995)).

Several strategies have been pursued to decrease viral-induced inflammatory responses. One strategy involves diminishing cytotoxic T lymphocyte (CTL) responses by minimizing viral gene expression through use of second- and third-generation adenoviral vectors in which multiple viral genetic loci are deleted or rendered defective by mutation (Christ et al, Hum. Gene Therapy 11: 415-427 (2000); Gao et al, J. Virol. 70: 8934-8943 (1996)). For the latter, adenoviral vectors with E1- and E4-deleted regions have been shown to express less viral protein and to exhibit substantially less toxicity in terms of vector-induced hepatitis (Gao et al, J. Virol. 70: 8934-8943 (1996); Wang et al, Gene Ther. 4: 393-400 (1997)). Another strategy involves the use of systemic corticosteroids (Sterman et al, Cancer Gene Therapy 7: 1511-1518 (2000)).

Accordingly, there is a need in the art of gene therapy for better control of transgene expression and inflammatory responses in subjects treated with recombinant viral vectors.

### SUMMARY OF THE INVENTION

One aspect described herein provides a method of reducing toxicity in treating a subject with a recombinant viral vector, such as a recombinant AAV (rAAV) vector, comprising co-administration of an antibiotic and viral vector to the subject.

Another aspect described herein provides a method for enabling administration of a viral vector, such as a rAAV vector, to a subject in the absence of prednisone, or in the presence of a low dose of prednisone, the method comprising co-administration of an antibiotic and at least 1.5e¹² viral vector. For example, the antibiotic is co-administered with at least 1.6e¹² viral vector.

The co-administration of the antibiotic can be (a) prior to administration of the viral vector; (b) at substantially the same time as administration of the viral vector; and/or (c) after administration of the viral vector.

In one embodiment of any aspect provided herein, the subject is further administered prednisone.

In one embodiment of any aspect provided herein, the subject is not administered prednisone.

In one embodiment of any aspect provided herein, the antibiotic is a member of the tetracycline family of antibiotics, or a member of the macrolides family of antibiotics. Exemplary members of the tetracycline family of antibiotics include Tetracycline, Chlortetracycline, Oxytetracycline, Demeclocycline, Lymecycline, Meclocycline, Methacycline, Minocycline, Rolitetracycline, Doxycycline, Tigecycline, Eravacycline, Sarecycline, and Omadacycline. Exemplary members of the macrolides family of antibiotics include of the Clarithromycin, Azithromycin, Fidoximycin, and Erythromycin.

In one embodiment of any aspect provided herein, the viral vector is administered at a dose of greater than 1.5e¹².

In one embodiment of any aspect provided herein, the viral vector genome, e.g., the rAAV vector genome, comprises 5′ and 3′ AAV inverted terminal repeats (ITR) sequences, and located between the 5′ and 3′ ITRs, a heterologous nucleic acid sequence encoding a therapeutic gene, wherein the heterologous nucleic acid is operatively linked to a promoter.

In one embodiment of any aspect provided herein, the viral vector comprises a capsid protein selected from the group consisting of hybrid, chimeric, mosaic, polyploid and haploid group of rAAVs.

In one embodiment of any aspect provided herein, the ITR is a wild-type or mutant ITR.

In one embodiment of any aspect provided herein, the therapeutic gene alters expression of a disease gene. In one embodiment of any aspect provided herein, the therapeutic gene increases or decreases expression of a disease gene. Exemplary disease genes include those listed herein in Table 1.

In one embodiment of any aspect provided herein, the subject is at risk of having, or has been diagnosed as having a disease selected of those diseases listed in Table 2.

In one embodiment of any aspect provided herein, wherein the antibiotic is administered at least twice. For example, that antibiotic is administered once daily for at least 16, 17, 18, 19, 20, 21, 22, 23, 24 or more weeks. The antibiotic can be administered periodically or by a pulsed regimen.

In one embodiment of any aspect provided herein, administration of the antibiotic and viral vector is systemic.

In one embodiment of any aspect provided herein, administration of the antibiotic is systemic and administration of the viral vector is local.

Another aspect described herein provides a composition comprising a recombinant viral vector, such as a rAAV vector, and an antibiotic.

In another aspect provided herein, tetracycline family of antibiotic is administered in combination with another antibiotic selected from either tetracycline family or the macrolides family of antibiotics.

Yet another aspect described herein provides a pharmaceutical composition comprising a recombinant viral vector, such as a rAAV vector, and an antibiotic.

In one embodiment of any aspect provided herein, the composition further comprises prednisone. In one embodiment of any aspect provided herein, the composition does not comprise prednisone.

### Definitions

The following terms are used in the description herein and the appended claims:

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of’ when used in a claim of this invention is not intended to be interpreted to be equivalent to “comprising.” Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

The term “parvovirus” as used herein encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, Muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of relatively new AAV serotypes and clades have been identified (see, e.g., Gao et al., (2004) J. Virology 78:6381-6388; Moris et al., (2004) Virology 33-:375-383; and Table 3).

The genomic sequences of various serotypes of AAV and the autonomous parvoviruses, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC 001829, NC 001862, NC 000883, NC_001701, NC 001510, NC_006152, NC_006261, AF063497, U89790, AF043303, AF028705, AF028704, J02275, J01901, J02275, X01457, AF288061, AH009962, AY028226, AY028223, NC 001358, NC_001540, AF513851, AF513852, AY530579; the disclosures of which are incorporated by reference herein for teaching parvovirus and AAV nucleic acid and amino acid sequences. See also, e.g., Srivistava et al., (1983) J Virology 45:555; Chiarini et al., (1998) J. Virology 71:6823; Chiarini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J Viral. 58:921; Gao et al., (2002) Proc. Nat. Acad. Sci. USA 99:11854; Morris et al., (2004) Virology 33-:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences.

The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV4 (Padron et al., (2005) J. Viral. 79: 5047-58), AAV5 (Walters et al., (2004) J. Viral. 78: 3361-71) and CPV (Xie et al., (1996) J. Mal. Biol. 6:497-520 and Tsao et al., (1991) Science 251: 1456-64).

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., enzyme replacement to reduce or eliminate symptoms of a disease, or improvement in transplant survivability or induction of an immune response.

By the terms “treat,” “treating” or “treatment of’ (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is substantially less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject).

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

An “rAAV vector genome” or “rAAV genome” is an AAV genome (i.e., vDNA) that comprises one or more heterologous nucleic acid sequences. rAAV vectors generally require only the inverted terminal repeat(s) (TR(s)) in cis to generate virus. All other viral sequences are dispensable and may be supplied in trans (Muzyczka, (1992) Curr. Topics Microbial. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV TR sequence), optionally two ITRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., an ITR that mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR,” including an “AAV inverted terminal repeat” or “AAV ITR” may be from any AAV, including but not limited to serotypes 1, 2, 3, 4, 5, 6, 7, 8, 9, 10, 11 or 12 or any other AAV now known or later discovered. An AAV terminal repeat need not have the native terminal repeat sequence (e.g., a native AAV TR or AAV ITR sequence may be altered by insertion, deletion, truncation and/or missense mutations), as long as the terminal repeat mediates the desired functions, e.g., replication, virus packaging, integration, and/or provirus rescue, and the like.

AAV proteins VP1, VP2 and VP3 are capsid proteins that interact together to form an AAV capsid of an icosahedral symmetry. VP1.5 is an AAV capsid protein described in US Publication No. 2014/0037585.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

A “chimeric’ capsid protein as used herein means an AAV capsid protein that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

As used herein, the term “haploid AAV” shall mean that AAV as described in U.S. patent application Ser. No. 16/051,110, which is incorporated herein.

The term a “hybrid” AAV vector or parvovirus refers to a rAAV vector where the viral TRs or ITRs and viral capsid are from different parvoviruses. Hybrid vectors are described in international patent publication WO 00/28004 and Chao et al., (2000) Molecular Therapy 2:619. For example, a hybrid AAV vector typically comprises the adenovirus 5′ and 3′ cis ITR sequences sufficient for adenovirus replication and packaging (i.e., the adenovirus terminal repeats and PAC sequence).

The term “polyploid AAV” refers to a AAV vector which is composed of capsids from two or more AAV serotypes, e.g., and can take advantages from individual serotypes for higher transduction but not in certain embodiments eliminate the tropism from the parents.

Additional patents incorporated for reference herein that are related to, disclose or describe an AAV or an aspect of an AAV, including the DNA vector that includes the gene of interest to be expressed are: U.S. Pat. Nos. 6,491,907; 7,229,823; 7,790,154; 7,201,898; 7,071,172; 7,892,809; 7,867,484; 8,889,641; 9,169,494; 9,169,492; 9,441,206; 9,409,953; and, 9,447,433; 9,592,247; and, 9,737,618.

A “promoter” is a nucleotide sequence which initiates and regulates transcription of a polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

“Operably linked” refers to an arrangement of elements wherein the components so described are configured so as to perform their usual function. Thus, a given promoter operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence. Thus, the term “operably linked” is intended to encompass any spacing or orientation of the promoter element and the DNA sequence of interest which allows for initiation of transcription of the DNA sequence of interest upon recognition of the promoter element by a transcription complex.

### DETAILED DESCRIPTION OF THE INVENTION

### Method of Treating a Toxicity

Provided herein is a method of reducing toxicity in treating a subject with recombinant viral vector, e.g., a recombinant AAV (rAAV) vector, the method comprising co-administration of an antibiotic and viral vector to the subject.

In one embodiment, toxicity is reduced by at least 5% following co-administration of the viral and antibiotic as compared to a reference level. In one embodiment, toxicity is reduced by at least about at least 1%, at least 2%, at least 3%, at least 4%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more following co-administration of the viral and antibiotic as compared to a reference level. As used herein, a “reference level” refers to the level of toxicity in an otherwise identical sample that is not co-administered an antibiotic, i.e., is only administered the viral. As used herein, “toxic” or ‘toxicity” refer to any observed clinically adverse effects related to administration of a viral vector, e.g., a viral vector, including but not limited to abnormal hematology or serum chemistry results, elevated immune response (e.g., increased chemokine secretion), an antigen specific T-cell response, and/or increased neutrophil production and/or activation. A variety of methods are available to one of ordinary skill in the art for the detection of toxicity, e.g., changes in an inflammatory response, including but not limited to: (1) protein assays (e.g. enzyme-linked immunosorbent assay (ELISA), such as the BioSource™ murine IL-6 ELISA kit; Western Blot analysis, Cytometric Bead Array (CBA, such as the BD™ Biosciences CBA Mouse Inflammation Kit); and multiplex assays (including technologies similar to those utilized in the Bio-Plex® or Luminex® multiplex suspension arrays)); enzyme specific assays, for example, assays determining levels and activities of, e.g., liver enzymes (2) DNA assays (e.g. Southern Blot and polymerase chain reaction (PCR, including quantitative-PCR (Q-PCR)); (3) RNA assays (e.g. Northern Blot analysis, and PCR-based assays (including reverse-transcriptase PCR, real time PCR, and Tagman); and (4) other assays directed to immune cell identity, function, or markers (e.g. immunofluorescent staining of cell surface molecules for Flow Cytometric Analysis (FACS); Cytotoxic T Lymphocyte (CTL) assays (e.g. 51CR release); enzyme-linked immunospot (ELISPOT) assays; and major histocompatibility complex (MHC)-peptide tetramer staining as well as assays directed to numerous other functions (e.g., macrophage activity, antigen-specific T cells, and other cell-based assays for biological response modifiers)).

Toxicity is often measured by elevated enzyme levels. In particular, elevated liver transaminase (or, aminotransferase) levels or, liver inflammation causing asymptomatic transient elevation of liver transaminases indicate toxicity or liver toxicity. Liver function tests including but not limiting to albumin, aspartate aminotransferase (AST) or, serum glutamic-oxaloacetic transaminase (SGOT), alanine aminotransferase (ALT) or, serum glutamic-pyruvic transaminase (SGPT), gamma-glutamyltransferase (GGT), gamma-glutamyl transpeptidase, lactate dehydrogenase (LDH), alkaline phosphatase (ALP) or, total bilirubin is performed while determining toxicity. Measurement of creatine kinase (CK) or creatine phosphokinase (CPK) are also included in toxicity tests. Liver toxicity and muscle toxicity are major contributors of toxicity in human body. Liver function tests are further described in, e.g., Gowda S. et al. PanAfrican Medical Journal (2009), the contents of which is incorporated herein by reference in its entirety.

Elevations of enzymes greater than 1.1 fold, greater than 1.2 fold, greater than 1.3 fold, greater than 1.4 fold, greater than 1.5 fold, greater than 1.6 fold, greater than 1.7 fold, greater than 1.8 fold, greater than 1.9 fold, or, greater than 2 fold the upper limit of normal enzyme level reflects toxicity. In certain embodiments the toxicity is indicated by elevations of enzymes greater than 2.5 fold, greater than 3 fold, greater than 3.5 fold, greater than 4 fold, greater than 4.5 fold, greater than 5 fold, greater than 5.5 fold, greater than 6 fold, greater than 6.5 fold, greater than 7 fold, greater than 7.5 fold, greater than 8 fold, greater than 8.5 fold, greater than 9 fold, greater than 9.5 fold, greater than 10 fold, greater than 11 fold, greater than 12 fold, greater than 13 fold, greater than 14 fold, greater than 15 fold, greater than 16 fold, greater than 17 fold, greater than 18 fold, greater than 19 fold, greater than 20 fold, greater than 25 fold, greater than 30 fold, greater than 40 fold, greater than 45 fold, greater than 50 fold, greater than 60 fold, greater than 70 fold, greater than 80 fold, greater than 90 fold, or greater than 100 fold the upper limit of normal enzyme level.

In some embodiments, the ratio of certain enzyme activities, for example, the ratio of AST activity to ALT activity (AST/ALT ratio) is used a diagnostic criteria of liver toxicity. In certain embodiments, liver toxicity is identified by AST/ALT ratio greater than 1.0, greater than 1.1, greater than 1.2, greater than 1.3, greater than 1.4, greater than 1.5, greater than 1.6, greater than 1.7, greater than 1.8, greater than 1.9, greater than 2.0, greater than 2.4, greater than 2.5, greater than 2.6, greater than 2.7, greater than 2.8, greater than 2.9, greater than 3.0, greater than 3.5, greater than 4, greater than 5 or more.

In certain embodiments, the toxicity is identified as elevated level of ALT and/or AST over normal level in conjunction with elevated level of bilirubin and/or alkaline phosphatase (ALP). The normal level is a range of values that are accepted in a standard clinical care. The range of normal values differs per tests and laboratories. In some embodiments, the toxicity is determined by standard clinical chemistry, standard hematology or histopathology methods.

In one embodiment, co-administration of the antibiotic prevents the occurrence or onset of toxicity associated with the administration of the viral vector, e.g., the rAAV vector.

Toxicity can be localized, but is not limited to, to a specific organ or tissue which expresses the viral vector, for example, administration of a liver-specific viral can result in liver-specific toxicity. Exemplary organs or tissues include, the liver (or specifically the liver right lobe, liver left lobe, liver median lobe, liver caudate lobe), spleen, Brain, Skeletal Muscle, Heart, Aorta, lungs, blood vessels, pancreas, bladder, reproductive system, small intestine, large intestine, esophagus, rectum, thyroid, diaphragm, stomach, kidney, or the like. Alternatively, expression of viral can result in toxicity in a tissue or organ in which it is not expressed, for example, administration of a liver-specific viral can result in kidney-specific toxicity.

In some embodiments, the administration of antibiotic alone or by co-administration of the antibiotic along with steroid e.g., prednisone as described in the present invention leads to significant reduction of toxicity e.g., significant reduction of liver enzymes. In some embodiments the administration of antibiotic alone or by co-administration of the antibiotic along with steroid e.g., prednisone as described in the present invention leads to reduction of liver enzymes by at least 1.1-fold, at least 1.2 fold, at least 1.3 fold, at least 1.4 fold, at least 1.5-fold, at least 1.6 fold, at least 1.7 fold, at least 1.8 fold, at least 1.9 fold, at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 25 fold, at least 30 fold, at least 40 fold, at least 45 fold, at least 50 fold, at least 60 fold, at least 70 fold, at least 80 fold, at least 90 fold, at least 100 fold, or greater than 100 fold as compared to those who did not receive any treatment of antibiotic or antibiotic combined with steroid.

In some embodiments, the administration of antibiotic alone or by co-administration of the antibiotic along with steroid e.g., prednisone as described in the present invention leads to reduction of liver toxicity e.g., reduction in AST/ALT ratio as compared to those who did not receive any treatment of antibiotic or antibiotic combined with steroid. In several embodiments, the administration of antibiotic alone or by co-administration of the antibiotic along with steroid e.g., prednisone as described in the present invention leads to reduction AST/ALT ratio e.g., AST/ALT ratio is less than 2, less than 1.8, less than 1.6, less than 1.4, less than 1.2, less than 1.1, less than 1, less than 0.9, less than 0.8, less than 0.7, less than 0.6, less than 0.5 or even less.

In one embodiment, the co-administration of the antibiotic is prior to administration of the viral vector, e.g., the rAAV vector; at substantially the same time as administration of the viral vector; and/or after administration of the viral vector. For example, the antibiotic may be administered at least 1 month prior to administering the viral vector (e.g., 1, 2, 3, 4, 5, 6, 7, 8, 9, 10, 11, 12 months or more); 1 week or less prior to administering the viral vector; 24 hours or less prior to administering the viral vector, 1 hour or less prior to administering the viral vector, and/or five minutes or less prior to administering the viral vector. Alternatively, the antibiotic is administered concurrently with the viral vector, e.g., the antibiotic is administered within the same composition as the viral vector, or in separate, independent compositions that are administered together. Further, the antibiotic may be administered five minutes or less after administration of the viral vector, 1 hour or less after administration of the viral vector, 24 hours or less after administration of the viral vector, 1 week or less after administration of the viral vector, or at least 1 month after administration of the viral vector (e.g., 1, 2, 3, 4, 5, 6, 7, 8, 9, 10, 11, 12 months or more).

Wherein the antibiotic is administered more than once, the administrations can be prior to, concurrently, or after administration of the viral vector. For example, the antibiotics and be administered concurrently with the viral vector, and following the viral vector.

Co-administration of a steroid, such as prednisone, with a viral vector (e.g., a rAAV vector) has been shown to reduce or inhibit the activated immune response resulting from high viral vector expression. However, long term administration of a prednisone has been shown to result in elevated pressure in the eyes (glaucoma) and clouding of the lens in one or both eyes (cataracts), high blood pressure, high blood sugar, memory, behavioral and other psychological effects, weight gain, increased susceptibility to bacterial, viral and fungal infections, osteoporosis, and/or suppressed adrenal gland hormone production. Thus, long term use of prednisone is not desired.

Accordingly, provided herein is a method for enabling administration of a viral vector, e.g., a rAAV vector, to a subject in the absence of prednisone, or in the presence of a low dose of prednisone, the method comprising co-administration of an antibiotic and at least 1.5e12 viral vector.

Additionally, provided herein is a method directed to treating a genetic disease comprising administering to a subject a viral and an antibiotic. In one embodiment, the viral vector administration is a high therapeutically effective dose. In one embodiment, the subject is further administered a steroid, e.g., prednisone. In one embodiment, the subject is not further administered a steroid. In one embodiment, the subject is not further administered prednisone.

In one embodiment, the low dose prednisone is 40 milligrams per day. In one embodiment, the low dose prednisone is at least 40 milligrams per day, for example, at least 45 milligrams per day, 50 milligrams per day, 55 milligrams per day, 60 milligrams per day or more per day.

In one embodiment, a steroid, e.g., prednisone, is further administered the subject receiving co-administration of an antibiotic and a viral vector. In one embodiment, the steroid is a corticosteroid. Exemplary corticosteroids include (1) hydrocortisone/cortisone; (2) prednisolone/prednisone/methylprednisolone; (3) betamethasone/dexamethasone; and (4) triamcinolone. In one embodiment, the steroid is selected from alclometasone, alclometasone dipropionate, amcinonide, augmented betamethasone, augmented betamethasone dipropionate, beclomethasone, beclomethasone dipropionate, betamethasone, betamethasone benzoate, betamethasone dipropionate, betamethasone sodium phosphate, betamethasone valerate, budesonide, clobetasol, clobetasol propionate, clocortolone, clocortolone pivalate, cortisone, desonide, desoximetasone, dexamethasone, dexamethasone acetate, dexamethasone sodium phosphate, diflorasone, diflorasone acetonide, diflorasone diacetate, flucinolone, fludroxycortide, flunisolide, fluocinolone acetonide, fluocinonide, flurandrenolide, fluticasone, fluticasone propionate, halcinonide, halobetasol, halobetasol propionate, hydrocortisone, hydrocortisone acetate, hydrocortisone butyrate, hydrocortisone sodium phosphate, hydrocortisone valerate, methylprednisolone, methylprednisolone acetate, methylprednisolone sodium succinate, mometasone, mometasone furoate, prednicarbate, prednisolone, prednisolone acetate, prednisolone sodium phosphate, prednisolone tebutate, prednisone, triamcinolone, triamcinolone acetonide, triamcinolone diacetate, tiamcinolone hexacetonide, ulobetasol, a combination of two or more of these steroids, or commercial products of these steroids.

In one embodiment, the prednisone is administered prior to co-administration of the viral vector and antibiotic; at substantially the same time as co-administration of the viral vector and antibiotic; and/or after co-administration of the viral vector and antibiotic. For example, the prednisone may be administered 1 week or less prior to co-administration of the viral vector and antibiotic; 24 hours or less prior to co-administration of the viral vector and antibiotic, 1 hour or less prior to co-administration of the viral vector and antibiotic, and/or five minutes or less prior to co-administration of the viral vector and antibiotic. Alternatively, the prednisone is administered concurrently with co-administration of the viral vector and antibiotic. Further, prednisone may be administered five minutes or less after co-administration of the viral vector and antibiotic, 1 hour or less after co-administration of the viral vector and antibiotic, 24 hours or less after co-administration of the viral vector and antibiotic, or 1 week or less after co-administration of the viral vector and antibiotic. In one embodiment, prednisone is comprised in the same composition as the viral and/or the antibiotic.

In one embodiment, a steroid, e.g., prednisone, is not administered the subject receiving co-administration of an antibiotic and a viral vector, e.g., a rAAV vector.

### Viral Vectors

In various embodiments, the methods described herein comprise co-administering a viral vector and an antibiotic. In one embodiment, the viral vector is a DNA or RNA virus. Nonlimiting examples of a viral vector of this invention include an AAV vector, an adenovirus vector, a lentivirus vector, a retrovirus vector, a herpesvirus vector, an alphavirus vector, a poxvirus vector, a baculovirus vector, and a chimeric virus vector.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Peaenation mosaic virus group; Phycodnaviridae; Picornaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

Viral vectors of the invention may comprise the genome, in part or entirety, of any naturally occurring and/or recombinant viral vector nucleotide sequence (e.g., AAV, AV, LV, etc.) or variant. Viral vector variants may have genomic sequences of significant homology at the nucleic acid and amino acid levels, produce viral vector which are generally physical and functional equivalents, replicate by similar mechanisms, and assemble by similar mechanisms.

Variant viral vector sequences can be used to deliver a synthetic nucleic acid in vivo as described herein. For example, one or more sequences having at least about 70%, at least about 75%, at least about 80%, at least about 85%, at least about 90%, at least about 95%, at least about 99%, or more nucleotide and/or amino acid sequence identity (e.g., a sequence having about 75-99% nucleotide sequence identity) to a given vector (for example, AAV, AV, LV, etc.).

It is understood that a viral vector would further comprise components necessary for the given vector. For example, typical production of an AAV requires the presence of at least one Replication (Rep) genes and/or at least Capsid (Cap) genes. On the left side of the AAV genome there are two promoters called p5 and p19, from which two overlapping messenger ribonucleic acids (mRNAs) of different length can be produced. Each of these contains an intron which can be either spliced out or not, resulting in four potential Rep genes; Rep78, Rep68, Rep52 and Rep40. Rep genes (specifically Rep 78 and Rep 68) bind the hairpin formed by the ITR in the self-priming act and cleave at the designated terminal resolution site, within the hairpin. They are necessary for the AAVS1-specific integration of the AAV genome. All four Rep proteins were shown to bind ATP and to possess helicase activity. The right side of a positive-sensed AAV genome encodes overlapping sequences of three capsid proteins, VP1, VP2 and VP3, which start from one promoter, designated p40. The cap gene produces an additional, non-structural protein called the Assembly-Activating Protein (AAP). This protein is produced from ORF2 and is essential for the capsid-assembly process. Necessary elements for manufacturing AAV vectors are known in the art, and can further be reviewed, e.g., in U.S. Pat. Nos. 5,478,745A; 5,622,856A; 5,658,776A; 6,440,742B1; 6,632,670B1; 6,156,303A; 8,007,780B2; 6,521,225B1; 7,629,322B2; 6,943,019B2; 5,872,005A; and U.S. Patent Application Numbers US 2017/0130245; US20050266567A1; US20050287122A1; the contents of each are incorporated herein by reference in their entireties. In various embodiments, nucleic acids expressing Rep and/or Cap genes are transformed using standard methods, for example, by a plasmid, a virus, a liposome, a microcapsule, a non-viral vector, or as naked DNA.

Further, in various embodiments herein, the compositions described herein comprise a viral vector and an antibiotic.

In one embodiment, the viral vector is a rAAV vector and the genome comprises 5′ and 3′ AAV inverted terminal repeats (ITR) sequences, and located between the 5′ and 3′ ITRs, a heterologous nucleic acid sequence encoding a therapeutic gene, wherein the heterologous nucleic acid is operatively linked to a promoter.

A suitable promoter can be selected from any of a number of promoters known to one of ordinary skill in the art. In one embodiment, a promoter is a cell-type specific promotor. In a further embodiment, a promoter is an inducible promotor. Inducible promoters are further described in, e.g., International Patent Application Nos WO2019/038544 and WO/2016/181171, which are incorporated herein. In one embodiment, a promotor is located upstream 5′ and is operatively linked to the heterologous nucleic acid sequence. In one embodiment, the promotor is a liver cell-type specific promotor, a heart muscle cell-type specific promoter, a neuron cell-type specific promoter, a nerve cell-type specific promoter, a muscle cell-type specific promoter or another cell-type specific promoter.

In some embodiments, a constitutive promoter can be selected from a group of constitutive promoters of different strengths and tissue specificity. Some examples of these promoters are set forth in Table 5. A viral vector genome, e.g., a rAAV vector genome, can include one or more constitutive promoters, such as viral promoters or promoters from mammalian genes that are generally active in promoting transcription. Examples of constitutive viral promoters are: Herpes Simplex virus (HSV) promoter, thymidine kinase (TK) promoter. Rous Sarcoma Virus (RSV) promoter, Simian Virus 40 (SV40) promoter, Mouse Mammary Tumor Virus (MMTV) promoter, Ad EIA promoter and cytomegalovirus (CMV) promoters. Examples of constitutive mammalian promoters include various housekeeping gene promoters, as exemplified by the p-actin promoter and the chicken beta-actin (CB) promoter.

In an embodiment, the promoter is a tissue-specific promoter. Examples of tissue specific promoters that may be used with the viral vector genomes of the invention include the creatine kinase promoter, the myogenin promoter, the alpha myosin heavy chain promoter, the myocyte specific enhancer factor 2 (MEF2) promoter, the myoD enhancer element, albumin, alpha-1-antitrypsin promoter and hepatitis B virus core protein promoters, wherein the hepatitis B virus core protein promoters are specific for liver cells.

In an embodiment, a promoter is an inducible promoter. Examples of suitable inducible promoters include those from genes such as cytochrome P450 genes, heat shock protein genes, metallothionein genes, and hormone-inducible genes, including the estrogen gene promoter. Another example of an inducible promoter is the tetVP16 promoter that is responsive to tetracycline.

Promoters in a viral vector genome according to the disclosure herein include, but are not limited to neuron-specific promoters, such as synapsin 1 (SYN) promoter; muscle creatine kinase (MCK) promoters; and desmin (DES) promoters. In one embodiment, the viral-mediated expression of heterologous nucleic acids (such as human GAA) can be achieved in neurons via a Synapsin promoter or in skeletal muscles via an MCK promoter. Other promoters that can be used include, EF, B19p6, CAG, neurone specific enolase gene promoter; chicken beta-actin/CMV hybrid promoter; platelet derived growth factor gene promoter; bGH, EF1a, CamKIIa, GFAP, RPE, ALB, TBG, MBP, MCK, TNT, aMHC, GFP, RFP, mCherry, CFP and YFP promoters.

TABLE 5
Exemplary promoters:

| Promoter | Description/Loci | | Target cell | | |
|---|---|---|---|---|---|
| name | (plasmid names) | Size | type | Notes | References |
| CMV | Cytomegalovirus | ~600 bps | most cell types | Can undergo | Zolotukhin |
| | immediate early | | | silencing in- | et al. 1996; |
| | promoter(pTR-UF5) | | | vivo | Zolotukhin |
| | | | | | et a. 1999 |
| CBAaka: CB, | Hybrid CMV/Chicken | 1720 bps | most cell types | Contains | Acland et |
| CAG | beta actin | | | 381 bps | al. 2001; |
| | promoter(pTR- UF11, | | | version of | Cideciyan |
| | pTR-UF-SB) | | | CMV i.e. | et al. 2008 |
| | | | | enhancer | |
| smCBAaka: | Truncated CBA | 953 bps | most cell types | Chimeric | Pang et al. |
| small CBA | promoter | | | Intron | 2008; |
| | | | | collapsed. Used | |
| | | | | for ScAAV | |
| MOPS aka: | Proximal murine | ~500 bps | Photoreceptors, | | Flannery et |
| mOP, mRHO, | rhodopsin promoter | | primarily rods | | al. 1997; |
| MOPS500 | | | | | |
| GRK1aka: | Human rhodopsin | 292 bps | Photoreceptor, | Does not | Khani et |
| hGRK, hRK, | kinase 1 promoter | | rods and cones | transduce | al. 2007; |
| RK1 | | | (mouse and | cones in dog | Boye et al. |
| | | | primate) | | 2010; |
| | | | | | Boye et al. |
| | | | | | 2012 |
| IRBPaka: | Human inter- | 241 bps | Photoreceptors, | | Beltran et |
| hIRBP241 | photoreceptor retinoid | | rods and cones | | al. 2012 |
| | binding | | (mouse and | | |
| | protein/Retinol- | | dog) | | |
| | binding protein 3 | | | | |
| PR2.1aka: | Human red opsin | ~2100 bps | L and M cones | | Alexander |
| CHOPS2053 | promoter | | | | et al. 2007; |
| | | | | | Mancuso |
| | | | | | et al. 2009; |
| | | | | | Komaromy |
| | | | | | et al. 2010 |
| IRBP/GNAT2 | hIRBP enhancer fused | 524 bps | L/M and S | Efficiently | |
| | to cone transducin | | cones | transduces all | |
| | alpha promoter | | | classes of | |
| | | | | cones | |
| VMD2Aka: | Human vitelliform | 625 bps | RPE | Highly | Deng et al. |
| BEST1 | macular | | | selective for | 2012 |
| | dystrophy/Bestrophin | | | RPE | |
| | 1 promoter | | | | |
| VEcadaka: | VE-cadherin/Cadherin | 2530 bps | Vascular | | Cai et al. |
| VEcadherin | 5 (CDH5)/CD144 | | endothelial | | 2011; Qi et |
| | promoter | | cells | | al. 2012 |

In some embodiments, the viral vector genome, e.g., the rAAV vector genome, described herein further includes at least one poly-A tail that is located 3′ and downstream from the heterologous nucleic acid sequence. In some embodiments, the polyA signal is 3′ of a stability sequence or CS sequence as defined herein. Any polyA sequence can be used, including but not limited to hGH poly A, synpA polyA and the like. In some embodiments, the polyA is a synthetic polyA sequence. In some embodiments, the viral vector genome comprises two poly-A tails, e.g., a hGH poly A sequence and another polyA sequence, where a spacer nucleic acid sequence is located between the two poly A sequences. In some embodiments, the first and second poly A sequence is a hGH poly A sequence, and in some embodiments, the first and second poly A sequences are a synthetic poly A sequence. In some embodiments, the first poly A sequence is a hGH poly A sequence and the second poly A sequence is a synthetic sequence, or vice versa—that is, in alternative embodiments, the first poly A sequence is a synthetic poly A sequence and the second poly A sequence is a hGH polyA sequence. An exemplary poly A sequence is, for example, See SEQ ID NO: 15 in Application No. US20140348794 (hGH poly A sequence, which is incorporated herein by reference), or a poly A nucleic acid sequence having at least 80%, 85%, 90%, 95%, 96%, 97%, 98% or 99% nucleotide sequence identity to SEQ ID NO: 15 in Application No. US20140348794. In some embodiments, the hGHpoly sequence encompassed for use is described in Anderson et al. J. Biol. Chem 264(14); 8222-8229, 1989 (See, e.g. p. 8223, 2nd column, first paragraph) which is incorporated herein in its entirety by reference.

In some embodiments, a poly-A tail can be engineered to stabilize the RNA transcript that is transcribed from an viral vector genome, including a transcript for a heterologous gene. In an alternative embodiment, the poly-A tail can be engineered to include elements that are destabilizing. In an embodiment, a poly-A tail can be engineered to become a destabilizing element by altering the length of the poly-A tail.

In all aspects of the methods and compositions as disclosed herein, the viral vector genome may also comprise a stuffer DNA nucleic sequence. A stuffer nucleic acid sequence (also referred to as a “spacer” nucleic acid fragment) can be located between the poly A sequence and the 3′ ITR (i.e., a stuffer nucleic acid sequence is located 3′ of the polyA sequence and 5′ of the 3′ ITR). Such a stuffer nucleic acid sequence can be about 30 bp, 50 bp, 75 bp, 100 bp, 150 bp, 200 bp, 250 bp, 300 bp or longer than 300 bp. In some embodiments of the methods and compositions as disclosed herein, a stuffer nucleic acid fragment is between 20-50 bp, 50-100 bp, 100-200 bp, 200-300 bp, 300-500 bp, or any integer between 20-500 bp.

AAV ITRs

The viral vector genome as disclosed herein is a rAAV vector genome that comprises AAV ITRs that have desirable characteristics and can be designed to modulate the activities of, and cellular responses to vectors that incorporate the ITRs. In another embodiment, the AAV ITRs are synthetic AAV ITRs that has desirable characteristics and can be designed to manipulate the activities of and cellular responses to vectors comprising one or two synthetic ITRs, including, as set forth in U.S. Pat. No. 9,447,433, which is incorporated herein by reference.

The AAV ITRs for use in the rAAV genome as disclosed herein may be of any serotype suitable for a particular application. In some embodiments, the AAV vector genome is flanked by AAV ITRs. In some embodiments, the rAAV vector genome is flanked by AAV ITRs, wherein an ITR comprises a full length ITR sequence, an ITR with sequences comprising CPG islands removed, an ITR with sequences comprising CPG sequences added, a truncated ITR sequence, an ITR sequence with one or more deletions within an ITR, an ITR sequence with one or more additions within an ITR, or a combination of comprising any portion of the aforementioned ITRs linked together to form a hybrid ITR.

In order to facilitate long term expression, the polynucleotide encoding a therapeutic gene is interposed between AAV inverted terminal repeats (ITRs) (e.g., the first or 5′ and second 3′ AAV ITRs). AAV ITRs are found at both ends of a WT rAAV vector genome, and serve as the origin and primer of DNA replication. ITRs are required in cis for AAV DNA replication as well as for rescue, or excision, from prokaryotic plasmids. In an embodiment, the AAV ITR sequences that are contained within the nucleic acid of the rAAV genome can be derived from any AAV serotype (e.g. 1, 2, 3, 3b, 4, 5, 6, 7, 8, 9, and 10) or can be derived from more than one serotype, including combining portions of two or more AAV serotypes to construct an ITR. In an embodiment, for use in the rAAV vector, including an rAAV vector genome, the first and second ITRs should include at least the minimum portions of a WT or engineered ITR that are necessary for packaging and replication.

In some embodiments, the rAAV vector genome comprises at least one AAV ITR, wherein said ITR comprises, consists essentially of, or consists of; (a) an AAV rep binding element; (b) an AAV terminal resolution sequence; and (c) an AAV RBE (Rep binding element); wherein said ITR does not comprise any other AAV ITR sequences. In another embodiment, elements (a), (b), and (c) are from an AAV2 ITR and the ITR does not comprise any other AAV2 ITR sequences. In a further embodiment, elements (a), (b) and (c) are from any AAV ITR, including but not limited to AAV2, AAV8 and AAV9. In some embodiments, the polynucleotide comprises two synthetic ITRs, which may be the same or different.

In some embodiments, the polynucleotide in the rAAV vector, including an rAAV vector genome comprises two ITRs, which may be the same or different. In some embodiments, the AAV ITRs are the exemplary ITRs presented in U.S. Pat. Nos. 7,790,154; 8,361,457; 8,784,799; 9,447,433; 9,169,494; or 10,233,428, which are incorporated herein by reference. In certain embodiments, the AAV ITR and the AAV cap genes are from different serotypes. In certain embodiments, the AAV ITR and AAV cap genes are from the same serotype. In some embodiments the AAV ITRs are wild type, mutant or synthetic. The three elements in the ITR have been determined to be sufficient for ITR function. This minimal functional ITR can be used in all aspects of AAV vector production and transduction. Additional deletions may define an even smaller minimal functional ITR. The shorter length advantageously permits the packaging and transduction of larger transgenic cassettes.

In one embodiment, the mutant ITR is a DD mutant ITR (DD-ITR). A DD-ITR has the same sequence the ITR from which it is derived, but includes a second D sequence adjacent the A sequence, so there are D and D′. The D and D′ can anneal (e.g., as described in U.S. Pat. No. 5,478,745, the contents of which are incorporated herein by reference). Each D is typically about 20 nt in length, but can be as small as 5 nucleotides. Shorter D regions preserve the A-D junction (e.g., are generated by deletions at the 3′ end that preserve the A-D junction). Preferably the D region retains the nicking site and/or the A-D junction. The DD-ITR is typically about 165 nucleotides. The DD-ITR has the ability to provide information in cis for replication of the DNA construct. Thus, a DD-ITR has an inverted palindromic sequence with flanking D and D′ elements, e.g. a (+) strand 5′ to 3′ sequence of 5′-DABB′CC′A′D′-3′ and a (−) strand complimentary to the (+) strand that has a 5′ to 3′ sequence of 5′-DACC′BB′A′D′-3′ that can form a Holiday structure. In certain embodiments, the DD-ITR may have deletions in its components (e.g. A-C), while still retaining the D and D′ element. In certain embodiments, the ITR comprises deletions while still retaining the ability to form a Holliday structure and retaining two copies of the D element (D and D′). The DD-ITR may be generated from a native AAV ITR or from a synthetic ITR. In certain embodiments, the deletion is in the B region element. In certain embodiments, the deletion is in the C region element. In certain embodiments, a deletion within both the B and C element of the ITR. In one embodiment, the entire B and/or C element is deleted, and e.g. replaced with a single hairpin element. In one embodiment, the template comprises at least two DD-ITRs.

In another embodiment, each of the elements that are present in a synthetic ITR can be the exact sequence as exists in a naturally occurring AAV ITR (the WT sequence) or can differ slightly (e.g., by mutation such as differ by addition, deletion, and/or substitution of 1, 2, 3, 4, 5 or more nucleotides) so long as the functioning of the elements of the AAV ITR continue to function at a level sufficient to are not substantially different from the functioning of these same elements as they exist in a naturally occurring AAV ITR. In certain embodiments, AAV ITR is synthetic.

In a further embodiment, rAAV vector, including an rAAV vector genome can comprise, between the ITRs, one or more additional non-AAV cis elements, e.g., elements that initiate transcription, mediate enhancer function, allow replication and symmetric distribution upon mitosis, or alter the persistence and processing of transduced genomes. Such elements are well known in the art and include, without limitation, promoters, enhancers, chromatin attachment sequences, telomeric sequences, cis-acting microRNAs (miRNAs), and combinations thereof.

In another embodiment, an ITR exhibits modified transcription activity relative to a naturally occurring ITR, e.g., ITR2 from AAV2. It is known that the ITR2 sequence inherently has promoter activity. It also inherently has termination activity, similar to a poly(A) sequence. The minimal functional ITR of the present invention exhibits transcription activity as shown in the examples, although at a diminished level relative to ITR2. Thus, in some embodiments, the ITR is functional for transcription. In other embodiments, the ITR is defective for transcription. In certain embodiments, the ITR can act as a transcription insulator, e.g., preventing transcription of a transgenic cassette present in the vector when the vector is integrated into a host chromosome.

In one embodiment, the rAAV vector genome comprising at least one synthetic AAV ITR, wherein the nucleotide sequence of one or more transcription factor binding sites in the ITR is deleted and/or substituted, relative to the sequence of a naturally occurring AAV ITR such as ITR2. In some embodiments, it is the minimal functional ITR in which one or more transcription factor binding sites are deleted and/or substituted. In some embodiments at least 1 transcription factor binding site is deleted and/or substituted, e.g., at least 5 or more or 10 or more transcription factor binding sites, e.g., at least 1, 2, 3, 4, 5, 6, 7, 8, 9, 10, 11, 12, 13, 14, 15, 16, 17, 18, 19, 20, or 21 transcription factor binding sites.

Another embodiment, a rAAV vector, including an rAAV vector genome as described herein comprises a polynucleotide comprising at least one synthetic AAV ITR, wherein one or more CpG islands (a cytosine base followed immediately by a guanine base (a CpG) in which the cytosines in such arrangement tend to be methylated) that typically occur at, or near the transcription start site in an ITR are deleted and/or substituted. In an embodiment, deletion or reduction in the number of CpG islands can reduce the immunogenicity of the rAAV vector. This results from a reduction or complete inhibition in TLR-9 binding to the rAAV vector DNA sequence, which occurs at CpG islands. It is also well known that methylation of CpG motifs results in transcriptional silencing. Removal of CpG motifs in the ITR is expected to result in decreased TLR-9 recognition and/or decreased methylation and therefore decreased transgene silencing. In some embodiments, it is the minimal functional ITR in which one or more CpG islands are deleted and/or substituted. In an embodiment, AAV ITR2 is known to contain 16 CpG islands of which one or more, or all 16 can be deleted.

In some embodiments, at least 1 CpG motif is deleted and/or substituted, e.g., at least 4 or more or 8 or more CpG motifs, e.g., at least 1, 2, 3, 4, 5, 6, 7, 8, 9, 10, 11, 12, 13, 14, 15, or 16 CpG motifs. The phrase “deleted and/or substituted” as used herein means that one or both nucleotides in the CpG motif is deleted, substituted with a different nucleotide, or any combination of deletions and substitutions.

In another embodiment, the synthetic ITR comprises, consists essentially of, or consists of one of the nucleotide sequences listed below. In other embodiments, the synthetic ITR comprises, consist essentially of, or consist of a nucleotide sequence that is at least 80% identical, e.g., at least 85%, 90%, 95%, 96%, 97%, 98%, or 99% identical to one of the nucleotide sequences listed below.

MH-257
(SEQ ID NO: 36)
AGGAACCCCTAGTGATGGAGTTGGCCACTCCCTCTCTGCGCGCTCGCTC
GCTCACTGAGGCAATTTGATAAAAATCGTCAAATTATAAACAGGCTTTG
CCTGTTTAGCCTCAGTGAGCGAGCGAGCGCGCAGAGAGGGAGTGGCCAA
CTCCATCACTAGGGGTTCCT
MH-258
(SEQ ID NO: 37)
AGGAACCCCTAGTGATGGAGTTGGCCACTCCCTCTCTGCGCGCTCGCTC
GCTCACTGAGGGATAAAAATCCAGGCTTTGCCTGCCTCAGTGAGCGAGC
GAGCGCGCAGAGAGGGAGTGGCCAACTCCATCACTAGGGGTTCCT
MH Delta 258
(SEQ ID NO: 38)
AGGAACCCCTAGTGATGGAGTTGGCCACTCCCTCTCTGCGCGCTCGCTC
GCTCACTGAGGGATAAAAATCCAGGCTTTGCCTGCCTCAGTGAGCGAGC
GAGCGCGCAGAGAGGGAGTGGCCAACTCCATCACTAGGGGTTCCT
MH Telomere-1 ITR
(SEQ ID NO: 39)
AGGAACCCCTAGTGATGGAGTTGGCCACTCCCTCTCTGGGATTGGGATT
GCGCGCTCGCTCGCGGGATTGGGATTGGGATTGGGATTGGGATTGGGAT
TGATAAAAATCAATCCCAATCCCAATCCCAATCCCAATCCCAATCCCGC
GAGCGAGCGCGCAATCCCAATCCCAGAGAGGGAGTGGCCAACTCCATCA
CTAGGGGTTCCT
MH Telomere-2 ITR
(SEQ ID NO: 40)
AGGAACCCCTAGTGATGGAGTTGGCCACTCCCTCTCTGCGCGCTCGCTC
GCTCGGGATTGGGATTGGGATTGGGATTGGGATTGGGATTGATAAAAAT
CAATCCCAATCCCAATCCCAATCCCAATCCCAATCCCGCGAGCGAGCGC
GCAGGAGAGGGAGTGGCCAACTCCATCACTAGGGGTTCCTAAGCTTATT
ATA
MH PolII 258 ITR
(SEQ ID NO: 41)
AGGAACCCCTAGTGATGGAGTTGGCCACTCCCTCTCTGCGCGCTCGCTC
GCTCACTGAGGGCGCCTATAAAGATAAAAATCCAGGCTTTGCCTGCCTC
AGTTAGCGAGCGAGCGCGCAGAGAGGGAGTGGCCAACTCCATCACTAGG
GGTTCCT
MH 258 Delta D conservative
(SEQ ID NO: 42)
CTAGTGATGGAGTTGGCCACTCCCTCTCTGCGCGCTCGCTCGCTCACTG
AGGGATAAAAATCCAGGCTTTGCCTGCCTCAGTGAGCGAGCGAGCGCGC
AGAGAGGGAGTGGCCAACTCCATCACTAG

In certain embodiments, a rAAV vector genome as described herein comprises a synthetic ITR that is capable of producing AAV virus particles that can transduce host cells. Such ITRs can be used, for example, for viral delivery of heterologous nucleic acids. Examples of such ITRs include MH-257, MH-258, and MH Delta 258 listed above.

In other embodiments, a rAAV vector genome as described herein containing a synthetic ITR is not capable of producing AAV virus particles. Such ITRs can be used, for example, for non-viral transfer of heterologous nucleic acids. Examples of such ITRs include MH Telomere-1, MH Telomere-2, and MH Pol II 258 listed above.

In a further embodiment, an rAAV vector genome as described herein comprising the synthetic ITR of the invention further comprises a second ITR which may be the same as or different from the first ITR. In one embodiment, an rAAV vector genome further comprises a heterologous nucleic acid, e.g., a sequence encoding a protein or a functional RNA. In an additional embodiment, a second ITR cannot be resolved by the Rep protein, i.e., resulting in a double stranded viral DNA.

In an embodiment, an rAAV vector genome comprises a polynucleotide comprising a synthetic ITR of the invention. In a further embodiment, the viral vector can be a parvovirus vector, e.g., an AAV vector. In another embodiment, a recombinant parvovirus particle (e.g., a recombinant AAV particle) comprises a synthetic ITR.

In one embodiment, the rAAV vector (also referred to as a rAAV virion) as disclosed herein comprises a capsid protein, and a rAAV genome in the capsid protein. A rAAV capsid of the rAAV virion used herein is any of those listed in Table 3, or any combination thereof.

TABLE 3
AAV Serotypes and exemplary Published corresponding capsid sequence

| Serotype and where capsid sequence is | Serotype and where capsid sequence is |
|---|---|
| published | published |
| AAV3.3b (See SEQ ID NO: 72 in US20030138772) | AAV3-3 (See SEQ ID NO: 200 US20150315612) |
| AAV3-3 (See SEQ ID NO: 217 US20150315612) | AAV3a ((See SEQ ID NO: 5 in US6156303) |
| AAV3a (See SEQ ID NO: 9 in US6156303) | AAV3b (See SEQ ID NO: 6 in US6156303) |
| AAV3b (See SEQ ID NO: 10 in US6156303) | AAV3b (See SEQ ID NO: 1 in US6156303) |
| AAV4 (See SEQ ID NO: 17 US20140348794) | AAV4 ((See SEQ ID NO: 5 in US20140348794) |
| AAV4 (See SEQ ID NO: 3 in US20140348794) | AAV4 (See SEQ ID NO: 14 in US20140348794) |
| AAV4 (See SEQ ID NO: 15 in US20140348794) | AAV4 (See SEQ ID NO: 19 in US20140348794) |
| AAV4 (See SEQ ID NO: 12 in US20140348794) | AAV4 (See SEQ ID NO: 13 in US20140348794) |
| AAV4 (See SEQ ID NO: 7 in US20140348794) | AAV4 (See SEQ ID NO: 8 in US20140348794) |
| AAV4 (See SEQ ID NO: 9 in US20140348794) | AAV4 (See SEQ ID NO: 2 in US20140348794) |
| AAV4 (See SEQ ID NO: 10 in US20140348794) | AAV4 (See SEQ ID NO: 11 in US20140348794) |
| AAV4 (See SEQ ID NO: 18 in US20140348794) | AAV4 (See SEQ ID NO: 63 in |
| | US20030138772) and US20160017295 SEQ |
| ID NO: (See SEQ ID NO: 4 in US20140348794) | AAV4 (See SEQ ID NO: 16 in US20140348794) |
| AAV4 (See SEQ ID NO: 20 in US20140348794) | AAV4 (See SEQ ID NO: 6 in US20140348794) |
| AAV4 (See SEQ ID NO: 1 in US20140348794) | AAV42.2 (See SEQ ID NO: 9 in US20030138772) |
| AAV42.2 (See SEQ ID NO: 102 in US20030138772) | AAV42.3b (See SEQ ID NO: 36 in US20030138772) |
| AAV42.3B (See SEQ ID NO: 107 in US20030138772) | AAV42.4 (See SEQ ID NO: 33 in US20030138772) |
| AAV42.4 (See SEQ ID NO: 88 in US20030138772) | AAV42.8 (See SEQ ID NO: 27 in US20030138772) |
| AAV42.8 (See SEQ ID NO: 85 in US20030138772) | AAV43.1 (See SEQ ID NO: 39 in US20030138772) |
| AAV43.1 (See SEQ ID NO: 92 in US20030138772) | AAV43.12 (See SEQ ID NO: 41 in US20030138772) |
| AAV43.12 (See SEQ ID NO: 93 in US20030138772) | AAV8 (See SEQ ID NO: 15 in US20150159173) |
| AAV8 (See SEQ ID NO: 7 in US20150376240) | AAV8 (See SEQ ID NO: 4 in US20030138772; |
| | US20150315612 SEQ |
| ID NO: 182 | AAV8 (See SEQ ID NO: 95 in |
| | US20030138772), US20140359799 SEQ |
| AAV8 (See SEQ ID NO: 31 in US20150159173) | AAV8 (See, e.g., SEQ ID NO: 8 in |
| | US20160017295, or SEQ ID NO: 7 in |
| | US7198951, or SEQ ID NO: 223 in |
| | US20150315612) |
| AAV8 (See SEQ ID NO: 8 in US20150376240) | AAV8 (See SEQ ID NO: 214 in US20150315612) |
| AAV-8b (See SEQ ID NO: 5 in US20150376240) | AAV-8b (See SEQ ID NO: 3 in US20150376240) |
| AAV-8h (See SEQ ID NO: 6 in US20150376240) | AAV-8h (See SEQ ID NO: 4 in US20150376240) |
| AAV9 (See SEQ ID NO: 5 in US20030138772) | AAV9 (See SEQ ID NO: 1 in US7198951) |
| AAV9 (See SEQ ID NO: 9 in US20160017295) | AAV9 (See SEQ ID NO: 100 in |
| | US20030138772), US7198951 SEQ ID NO: 2 |
| | AAV9 (See SEQ ID NO: 3 in US7198951) |
| AAV9 (AAVhu.14) (See SEQ ID NO: 3 in | AAV9 (AAVhu.14) (See SEQ ID NO: 123 in |
| US20150315612) | US20150315612) |
| AAVA3.1 (See SEQ ID NO: 120 in | AAVA3.3 (See SEQ ID NO: 57 in |
| US20030138772) | US20030138772) |
| AAVA3.3 (See SEQ ID NO: 66 in | AAVA3.4 (See SEQ ID NO: 54 in |
| US20030138772) | US20030138772) |
| AAVA3.4 (See SEQ ID NO: 68 in | AAVA3.5 (See SEQ ID NO: 55 in |
| US20030138772) | US20030138772) |
| AAVA3.5 (See SEQ ID NO: 69 in | AAVA3.7 (See SEQ ID NO: 56 in |
| US20030138772) | US20030138772) |
| AAVA3.7 (See SEQ ID NO: 67 in | AAV29. (See SEQ ID NO: 11 in (AAVbb. l) |
| US20030138772) | 161 US20030138772) |
| AAVC2 (See SEQ ID NO: 61 in US20030138772) | AAVCh.5 (See SEQ ID NO: 46 in |
| | US20150159173); US20150315612 SEQ |
| ID NO: 234 | AAVcy.2 (AAV13.3) (See SEQ ID NO: 15 in |
| | US20030138772) |
| AAV24.1 (See SEQ ID NO: 101 in | AAVcy.3 (AAV24.1) (See SEQ ID NO: 16 in |
| US20030138772) | US20030138772) |
| AAV27.3 (See SEQ ID NO: 104 in | AAVcy.4 (AAV27.3) (See SEQ ID NO: 17 in |
| US20030138772) | US20030138772) |
| AAVcy.5 (See SEQ ID NO: 227 in | AAV7.2 (See SEQ ID NO: 103 in |
| US20150315612) | US20030138772) |
| AAVcy.5 (AAV7.2) (See SEQ ID NO: 18 in | AAV16.3 (See SEQ ID NO: 105 in |
| US20030138772) | US20030138772) |
| AAVcy.6 (AAV16.3) (See SEQ ID NO: 10 in | AAVcy.5 (See SEQ ID NO: 8 in |
| US20030138772) | US20150159173) |
| AAVcy.5 (See SEQ ID NO: 24 in | AAVCy.5Rl (See SEQ ID NO: in |
| US20150159173) | US20150159173 |
| AAVCy.5R2 (See SEQ ID NO: in | AAVCy.5R3 (See SEQ ID NO: in |
| US20150159173) | US20150159173 |
| AAVCy.5R4 (See SEQ ID NO: in | AAVDJ (See SEQ ID NO: 3 in |
| US20150159173) | US20140359799) and SEQ ID NO: 2 in |
| | US7588772) |
| | AAVDJ (See SEQ ID NO: 2 in |
| | US20140359799; and SEQ ID NO: 1 in |
| | US7588772) |
| | AAVDJ-8 (See SEQ ID NO: in US7588772; |
| | Grimm et al. 2008 |
| AAVDJ-8 (See SEQ ID NO: in US7588772; | AAVF5 (See SEQ ID NO: 110 in |
| Grimm et al. 2008 | US20030138772) |
| AAVH2 (See SEQ ID NO: 26 in US20030138772) | AAVH6 (See SEQ ID NO: 25 in |
| | US20030138772) |
| AAVhEl.l (See SEQ ID NO: 44 in US9233131) | AAVhErl.14 (See SEQ ID NO: 46 in |
| | US9233131) |
| AAVhErl.16 (See SEQ ID NO: 48 in US9233131) | AAVhErl.18 (See SEQ ID NO: 49 in |
| | US9233131) |
| AAVhErl.23 (AAVhEr2.29) (See SEQ ID NO: 53 | AAVhErl.35 (See SEQ ID NO: 50 in |
| in US9233131) | US9233131) |
| AAVhErl.36 (See SEQ ID NO: 52 in US9233131) | AAVhErl.5 (See SEQ ID NO: 45 in |
| | US9233131) |
| AAVhErl.7 (See SEQ ID NO: 51 in US9233131) | AAVhErl.8 (See SEQ ID NO: 47 in |
| | US9233131) |
| AAVhEr2.16 (See SEQ ID NO: 55 in US9233131) | AAVhEr2.30 (See SEQ ID NO: 56 in |
| | US9233131) |
| AAVhEr2.31 (See SEQ ID NO: 58 in US9233131) | AAVhEr2.36 (See SEQ ID NO: 57 in |
| | US9233131) |
| AAVhEr2.4 (See SEQ ID NO: 54 in US9233131) | AAVhEr3.1 (See SEQ ID NO: 59 in |
| | US9233131) |
| AAVhu.l (See SEQ ID NO: 46 in US20150315612) | AAVhu.l (See SEQ ID NO: 144 in |
| | US20150315612) |
| AAVhu.lO (AAV16.8) (See SEQ ID NO: 56 in | AAVhu.lO (AAV16.8) (See SEQ ID NO: 156 |
| US20150315612) | in US20150315612) |
| AAVhu.ll (AAV16.12) (See SEQ ID NO: 57 in | AAVhu.ll (AAV16.12) (See SEQ ID NO: 153 |
| US20150315612) | in US20150315612) |
| AAVhu.12 (See SEQ ID NO: 59 in | AAVhu.12 (See SEQ ID NO: 154 in |
| US20150315612) | US20150315612) |
| AAVhu.13 (See SEQ ID NO: 16 in | |
| US2015015917 and ID NO: 71 in US20150315612) | |
| AAVhu.13 (See SEQ ID NO: 32 in | |
| US20150159173 and ID NO: 129 US20150315612) | |
| AAVhu.136.1 (See SEQ ID NO: 165 in | AAVhu.140.1 (See SEQ ID NO: 166 in |
| US20150315612) | US20150315612) |
| AAVhu.140.2 (See SEQ ID NO: 167 in | AAVhu.145.6 (See SEQ ID NO: 178 in |
| US20150315612) | US20150315612) |
| AAVhu.15 (See SEQ ID NO: 147 in | AAVhu.15 (AAV33.4) (See SEQ ID NO: 50 in |
| US20150315612) | US20150315612) |
| AAVhu.156.1 (See SEQ ID NO: 179 in | AAVhu.16 (See SEQ ID NO: 148 in |
| US20150315612) | US20150315612) |
| AAVhu.l6 (AAV33.8) (See SEQ ID NO: 51 in | AAVhu.17 (See SEQ ID NO: 83 in |
| US20150315612) | US20150315612) |
| AAVhu.l7 (AAV33.12) (See SEQ ID NO: 4 in | AAVhu.172.1 (See SEQ ID NO: 171 in |
| US20150315612) | US20150315612) |
| AAVhu.172.2 (See SEQ ID NO: 172 in | AAVhu.173.4 (See SEQ ID NO: 173 in |
| US20150315612) | US20150315612) |
| AAVhu.173.8 (See SEQ ID NO: 175 in | AAVhu.18 (See SEQ ID NO: 52 in |
| US20150315612) | US20150315612) |
| AAVhu.18 (See SEQ ID NO: 149 in | AAVhu.19 (See SEQ ID NO: 62 in |
| US20150315612) | US20150315612) |
| AAVhu.19 (See SEQ ID NO: 133 in | AAVhu.2 (See SEQ ID NO: 48 in |
| US20150315612) | US20150315612) |
| AAVhu.2 (See SEQ ID NO: 143 in | AAVhu.20 (See SEQ ID NO: 63 in |
| US20150315612) | US20150315612) |
| AAVhu.20 (See SEQ ID NO: 134 in | AAVhu.21 (See SEQ ID NO: 65 in |
| US20150315612) | US20150315612) |
| AAVhu.21 (See SEQ ID NO: 135 in | AAVhu.22 (See SEQ ID NO: 67 in |
| US20150315612) | US20150315612) |
| AAVhu.22 239 (See SEQ ID NO: 138 in | AAVhu.23 (See SEQ ID NO: 60 in |
| US20150315612) | US20150315612) |
| AAVhu.23.2 (See SEQ ID NO: 137 in | AAVhu.24 (See SEQ ID NO: 66 in |
| US20150315612) | US20150315612) |
| AAVhu.24 (See SEQ ID NO: 136 in | AAVhu.25 (See SEQ ID NO: 49 in |
| US20150315612) | US20150315612) |
| AAVhu.25 (See SEQ ID NO: 146 in | AAVhu.26 (See SEQ ID NO: 17 in |
| US20150315612) | US20150159173 and SEQ ID NO: 61 in |
| | US20150315612) |
| | AAVhu.26 (See SEQ ID NO: 33 in |
| | US20150159173), US20150315612 SEQ |
| | AAVhu.27 (See SEQ ID NO: 64 in |
| | US20150315612) |
| AAVhu.27 (See SEQ ID NO: 140 in | AAVhu.28 (See SEQ ID NO: 68 in |
| US20150315612) | US20150315612) |
| AAVhu.28 (See SEQ ID NO: 130 in | AAVhu.29 (See SEQ ID NO: 69 in |
| US20150315612) | US20150315612) |
| AAVhu.29 (See SEQ ID NO: 42 in | |
| US20150159173 and SEQ ID NO: 132 in | |
| US20150315612) | |
| AAVhu.29 (See SEQ ID NO: 225 in | AAVhu.29R (See SEQ ID NO: in |
| US20150315612) | US20150159173 |
| AAVhu.3 (See SEQ ID NO: 44 in | AAVhu.3 (See SEQ ID NO: 145 in |
| US20150315612) | US20150315612) |
| AAVhu.30 (See SEQ ID NO: 70 in | AAVhu.30 (See SEQ ID NO: 131 in |
| US20150315612) | US20150315612) |
| AAVhu.31 (See SEQ ID NO: 1 in | AAVhu.31 (See SEQ ID NO: 121 in |
| US20150315612) | US20150315612) |
| AAVhu.32 (See SEQ ID NO: 2 in | AAVhu.32 (See SEQ ID NO: 122 in |
| US20150315612) | US20150315612) |
| AAVhu.33 (See SEQ ID NO: 75 in | AAVhu.33 (See SEQ ID NO: 124 in |
| US20150315612) | US20150315612) |
| AAVhu.34 (See SEQ ID NO: 72 in | AAVhu.34 (See SEQ ID NO: 125 in |
| US20150315612) | US20150315612) |
| AAVhu.35 (See SEQ ID NO: 73 in | AAVhu.35 (See SEQ ID NO: 164 in |
| US20150315612) | US20150315612) |
| AAVhu.36 (See SEQ ID NO: 74 in | AAVhu.36 (See SEQ ID NO: 126 in |
| US20150315612) | US20150315612) |
| AAVhu.37 (See SEQ ID NO: 34 in | |
| US20150159173 and SEQ ID NO: 88 in | |
| US20150315612) | |
| AAVhu.37 (AAV106.1) (See SEQ ID NO: 10 in | |
| US20150315612 and SEQ ID NO: 18 in | |
| US20150159173) | |
| AAVhu.38 (See SEQ ID NO: 161 in | AAVhu.39 (See SEQ ID NO: 102 in |
| US20150315612) | US20150315612) |
| AAVhu.39 (AAVLG-9) (See SEQ ID NO: 24 in | AAVhu.4 (See SEQ ID NO: 47 in |
| US20150315612) | US20150315612) |
| AAVhu.4 (See SEQ ID NO: 141 in | AAVhu.40 (See SEQ ID NO: 87 in |
| US20150315612) | US20150315612) |
| AAVhu.40 (AAV114.3) (See SEQ ID NO: 11 in | AAVhu.41 (See SEQ ID NO: 91 in |
| US20150315612) | US20150315612) |
| AAVhu.41 (AAV127.2) (See SEQ ID NO: 6 in | AAVhu.42 (See SEQ ID NO: 85 in |
| US20150315612) | US20150315612) |
| AAVhu.42 (AAV127.5) (See SEQ ID NO:8 in | AAVhu.43 (See SEQ ID NO: 160 in |
| US20150315612) | US20150315612) |
| AAVhu.43 (See SEQ ID NO: 236 in | AAVhu.43 (AAV128.1) (See SEQ ID NO: 80 |
| US20150315612) | in US20150315612) |
| AAVhu.44 (See SEQ ID NO: 45 in | |
| US20150159173 and SEQ ID NO: 158 in | |
| US20150315612) | |
| AAVhu.44 (AAV128.3) (See SEQ ID NO: 81 in | AAVhu.44Rl (See SEQ ID NO: in |
| US20150315612) | US20150159173 |
| AAVhu.44R2 (See SEQ ID NO: in | AAVhu.44R3 (See SEQ ID NO: in |
| US20150159173 | US20150159173 |
| AAVhu.45 (See SEQ ID NO: 76 in | AAVhu.45 (See SEQ ID NO: 127 in |
| US20150315612) | US20150315612) |
| AAVhu.46 (See SEQ ID NO: 82 in | AAVhu.46 (See SEQ ID NO: 159 in |
| US20150315612) | US20150315612) |
| AAVhu.46 (See SEQ ID NO: 224 in | AAVhu.47 (See SEQ ID NO: 77 in |
| US20150315612) | US20150315612) |
| AAVhu.47 (See SEQ ID NO: 128 in | AAVhu.48 (See SEQ ID NO: 38 in |
| US20150315612) | US20150159173) |
| AAVhu.48 (See SEQ ID NO: 157 in | AAVhu.48 (AAV130.4) (See SEQ ID NO: 78 |
| US20150315612) | in US20150315612) |
| AAVhu.48Rl (See SEQ ID NO: in | AAVhu.48R2 (See SEQ ID NO: in |
| US20150159173 | US20150159173 |
| AAVhu.48R3 (See SEQ ID NO: in | AAVhu.49 (See SEQ ID NO: 209 in |
| US20150159173 | US20150315612) |
| AAVhu.49 (See SEQ ID NO: 189 in | AAVhu.5 (See SEQ ID NO: 45 in |
| US20150315612) | US20150315612) |
| AAVhu.5 (See SEQ ID NO: 142 in | AAVhu.51 (See SEQ ID NO: 208 in |
| US20150315612) | US20150315612) |
| AAVhu.51 (See SEQ ID NO: 190 in | AAVhu.52 (See SEQ ID NO: 210 in |
| US20150315612) | US20150315612) |
| AAVhu.52 (See SEQ ID NO: 191 in | AAVhu.53 (See SEQ ID NO: 19 in |
| US20150315612) | US20150159173) |
| AAVhu.53 (See SEQ ID NO: 35 in | AAVhu.53 (AAV145.1) (See SEQ ID NO: 176 |
| US20150159173) | in US20150315612) |
| AAVhu.54 (See SEQ ID NO: 188 in | AAVhu.54 (AAV145.5) (See SEQ ID NO: 177 |
| US20150315612) | in US20150315612) |
| AAVhu.55 (See SEQ ID NO: 187 in | AAVhu.56 (See SEQ ID NO: 205 in |
| US20150315612) | US20150315612) |
| AAVhu.56 (AAV145.6) (See SEQ ID NO: 168 in | AAVhu.56 (AAV145.6) (See SEQ ID NO: 192 |
| US20150315612) | in US20150315612) |
| AAVhu.57 (See SEQ ID NO: 206 in | AAVhu.57 (See SEQ ID NO: 169 in |
| US20150315612) | US20150315612) |
| AAVhu.57 (See SEQ ID NO: 193 in | AAVhu.58 (See SEQ ID NO: 207 in |
| US20150315612) | US20150315612) |
| AAVhu.58 (See SEQ ID NO: 194 in | AAVhu.6 (AAV3.1) (See SEQ ID NO: 5 in |
| US20150315612) | US20150315612) |
| AAVhu.6 (AAV3.1) (See SEQ ID NO: 84 in | AAVhu.60 (See SEQ ID NO: 184 in |
| US20150315612) | US20150315612) |
| AAVhu.60 (AAV161.10) (See SEQ ID NO: 170 in | AAVhu.6l (See SEQ ID NO: 185 in |
| US20150315612) | US20150315612) |
| AAVhu.61 (AAV161.6) (See SEQ ID NO: 174 in | AAVhu.63 (See SEQ ID NO: 204 in |
| US20150315612) | US20150315612) |
| AAVhu.63 (See SEQ ID NO: 195 in | AAVhu.64 (See SEQ ID NO: 212 in |
| US20150315612) | US20150315612) |
| AAVhu.64 (See SEQ ID NO: 196 in | AAVhu.66 (See SEQ ID NO: 197 in |
| US20150315612) | US20150315612) |
| AAVhu.67 (See SEQ ID NO: 215 in | AAVhu.67 (See SEQ ID NO: 198 in |
| US20150315612) | US20150315612) |
| AAVhu.7 (See SEQ ID NO: 226 in | AAVhu.7 (See SEQ ID NO: 150 in |
| US20150315612) | US20150315612) |
| AAVhu.7 (AAV7.3) (See SEQ ID NO: 55 in | AAVhu.71 (See SEQ ID NO: 79 in |
| US20150315612) | US20150315612) |
| AAVhu.8 (See SEQ ID NO: 53 in | AAVhu.8 (See SEQ ID NO: 12 in |
| US20150315612) | US20150315612) |
| AAVhu.8 (See SEQ ID NO: 151 in | AAVhu.9 (AAV3.1) (See SEQ ID NO: 58 in |
| US20150315612) | US20150315612) |
| AAVhu.9 (AAV3.1) (See SEQ ID NO: 155 in | AAV-LK01 (See SEQ ID NO: 2 in |
| US20150315612) | US20150376607) |
| AAV-LK01 (See SEQ ID NO: 29 in | AAV-LK02 (See SEQ ID NO: 3 in |
| US20150376607) | US20150376607) |
| AAV-LK02 (See SEQ ID NO: 30 in | AAV-LK03 (See SEQ ID NO: 4 in |
| US20150376607) | US20150376607) |
| AAV-LK03 (See SEQ ID NO: 12 in | |
| WO2015121501 and SEQ ID NO: 31 in | |
| US20150376607) | |
| AAV-LK04 (See SEQ ID NO: 5 in | AAV-LK04 (See SEQ ID NO: 32 in |
| US20150376607) | US20150376607) |
| AAV-LK05 (See SEQ ID NO: 6 in | AAV-LK05 (See SEQ ID NO: 33 in |
| US20150376607) | US20150376607) |
| AAV-LK06 (See SEQ ID NO: 7 in | AAV-LK06 (See SEQ ID NO: 34 in |
| US20150376607) | US20150376607) |
| AAV-LK07 (See SEQ ID NO: 8 in | AAV-LK07 (See SEQ ID NO: 35 in |
| US20150376607) | US20150376607) |
| AAV-LK08 (See SEQ ID NO: 9 in | AAV-LK08 (See SEQ ID NO: 36 in |
| US20150376607) | US20150376607) |
| AAV-LK09 (See SEQ ID NO: 10 in | AAV-LK09 (See SEQ ID NO: 37 in |
| US20150376607) | US20150376607) |
| AAV-LK10 (See SEQ ID NO: 11 in | AAV-LK10 (See SEQ ID NO: 38 in |
| US20150376607) | US20150376607) |
| AAV-LK11 (See SEQ ID NO: 12 in | AAV-LK11 (See SEQ ID NO: 39 in |
| US20150376607) | US20150376607) |
| AAV-LK12 (See SEQ ID NO: 13 in | AAV-LK12 (See SEQ ID NO: 40 in |
| US20150376607) | US20150376607) |
| AAV-LK13 (See SEQ ID NO: 14 in | AAV-LK13 (See SEQ ID NO: 41 in |
| US20150376607) | US20150376607) |
| AAV-LK14 (See SEQ ID NO: 15 in | AAV-LK14 (See SEQ ID NO: 42 in |
| US20150376607) | US20150376607) |
| AAV-LK15 (See SEQ ID NO: 16 in | AAV-LK15 (See SEQ ID NO: 43 in |
| US20150376607) | US20150376607) |
| AAV-LK16 (See SEQ ID NO: 17 in | AAV-LK16 (See SEQ ID NO: 44 in |
| US20150376607) | US20150376607) |
| AAV-LK17 (See SEQ ID NO: 18 in | AAV-LK17 (See SEQ ID NO: 45 in |
| US20150376607) | US20150376607) |
| AAV-LK18 (See SEQ ID NO: 19 in | AAV-LK18 (See SEQ ID NO: 46 in |
| US20150376607) | US20150376607) |
| AAV-LK19 (See SEQ ID NO: 20 in | AAV-LK19 (See SEQ ID NO: 47 in |
| US20150376607) | US20150376607) |
| AAV-PAEC (See SEQ ID NO: 1 in | AAV-PAEC (See SEQ ID NO: 48 in |
| US20150376607) | US20150376607) |
| AAV-PAEC11 (See SEQ ID NO: 26 in | AAV-PAEC11 (See SEQ ID NO: 54 in |
| US20150376607) | US20150376607) |
| AAV-PAEC 12 (See SEQ ID NO: 27 in | AAV-PAEC 12 (See SEQ ID NO: 51 in |
| US20150376607) | US20150376607) |
| AAV-PAEC 13 (See SEQ ID NO: 28 in | AAV-PAEC 13 (See SEQ ID NO: 49 in |
| US20150376607) | US20150376607) |
| AAV-PAEC2 (See SEQ ID NO: 21 in | AAV-PAEC2 (See SEQ ID NO: 56 in |
| US20150376607) | US20150376607) |
| AAV-PAEC4 (See SEQ ID NO: 22 in | AAV-PAEC4 (See SEQ ID NO: 55 in |
| US20150376607) | US20150376607) |
| AAV-PAEC6 (See SEQ ID NO: 23 in | AAV-PAEC6 (See SEQ ID NO: 52 in |
| US20150376607) | US20150376607) |
| AAV-PAEC7 (See SEQ ID NO: 24 in | AAV-PAEC7 (See SEQ ID NO: 53 in |
| US20150376607) | US20150376607) |
| AAV-PAEC8 (See SEQ ID NO: 25 in | AAV-PAEC8 (See SEQ ID NO: 50 in |
| US20150376607) | US20150376607) |
| AAVpi.l (See SEQ ID NO: 28 in US20150315612) | AAVpi.l (See SEQ ID NO: 93 in |
| | US20150315612; AAVpi.2 408, see SEQ ID |
| | NO: 30 in US20150315612) |
| AAVpi.2 (See SEQ ID NO: 95 in | AAVpi.3 (See SEQ ID NO: 29 in |
| US20150315612) | US20150315612) |
| AAVpi.3 (See SEQ ID NO: 94 in | AAVrh.10 (See SEQ ID NO: 9 in |
| US20150315612) | US20150159173) |
| AAVrh.10 (See SEQ ID NO: 25 in | AAV44.2 (See SEQ ID NO: 59 in |
| US20150159173) | US20030138772) |
| AAVrh.10 (AAV44.2) (See SEQ ID NO: 81 in | AAV42.1B (See SEQ ID NO: 90 in |
| US20030138772) | US20030138772) |
| AAVrh.l2 (AAV42.1b) (See SEQ ID NO: 30 in | AAVrh.13 (See SEQ ID NO: 10 in |
| US20030138772) | US20150159173) |
| AAVrh.13 (See SEQ ID NO: 26 in | AAVrh.13 (See SEQ ID NO: 228 in |
| US20150159173) | US20150315612) |
| AAVrh.l3R (See SEQ ID NO: in US20150159173 | AAV42.3A (See SEQ ID NO: 87 in |
| | US20030138772) |
| AAVrh.l4 (AAV42.3a) (See SEQ ID NO: 32 in | AAV42.5A (See SEQ ID NO: 89 in |
| US20030138772) | US20030138772) |
| AAVrh.l7 (AAV42.5a) (See SEQ ID NO: 34 in | AAV42.5B (See SEQ ID NO: 91 in |
| US20030138772) | US20030138772) |
| AAVrh.l8 (AAV42.5b) (See SEQ ID NO: 29 in | AAV42.6B (See SEQ ID NO: 112 in |
| US20030138772) | US20030138772) |
| AAVrh.l9 (AAV42.6b) (See SEQ ID NO: 38 in | AAVrh.2 (See SEQ ID NO: 39 in |
| US20030138772) | US20150159173) |
| AAVrh.2 (See SEQ ID NO: 231 in | AAVrh.20 (See SEQ ID NO: 1 in |
| US20150315612) | US20150159173) |
| AAV42.10 (See SEQ ID NO: 106 in | AAVrh.21 (AAV42.10) (See SEQ ID NO: 35 |
| US20030138772) | in US20030138772) |
| AAV42.11 (See SEQ ID NO: 108 in | AAVrh.22 (AAV42.11) (See SEQ ID NO: 37 |
| US20030138772) | in US20030138772) |
| AAV42.12 (See SEQ ID NO: 113 in | AAVrh.23 (AAV42.12) (See SEQ ID NO: 58 |
| US20030138772) | in US20030138772) |
| AAV42.13 (See SEQ ID NO: 86 in | AAVrh.24 (AAV42.13) (See SEQ ID NO: 31 |
| US20030138772) | in US20030138772) |
| AAV42.15 (See SEQ ID NO: 84 in | AAVrh.25 (AAV42.15) (See SEQ ID NO: 28 |
| US20030138772) | in US20030138772) |
| AAVrh.2R (See SEQ ID NO: in US20150159173 | AAVrh.31 (AAV223.1) (See SEQ ID NO: 48 |
| | in US20030138772) |
| AAVC1 (See SEQ ID NO: 60 in US20030138772) | AAVrh.32 (AAVC1) (See SEQ ID NO: 19 in |
| | 446 US20030138772) |
| AAVrh.32/33 (See SEQ ID NO: 2 in | AAVrh.51 (AAV2-5) (See SEQ ID NO: 104 in |
| US20150159173) | US20150315612) |
| AAVrh.52 (AAV3-9) (See SEQ ID NO: 18 in | AAVrh.52 (AAV3-9) (See SEQ ID NO: 96 in |
| US20150315612) | US20150315612) |
| AAVrh.53 (See SEQ ID NO: in US20150315612) | AAVrh.53 (AAV3-11) (See SEQ ID NO: 17 in |
| | US20150315612) |
| AAVrh.53 (AAV3-11) (See SEQ ID NO: 186 in | AAVrh.54 (See SEQ ID NO: 40 in |
| US20150315612) | US20150315612) |
| AAVrh.54 (See SEQ ID NO: 49 in | |
| US20150159173 and SEQ ID NO: 116 in | |
| US20150315612) | |
| AAVrh.55 (See SEQ ID NO: 37 in | AAVrh.55 (AAV4-19) (See SEQ ID NO: 117 |
| US20150315612) | in US20150315612) |
| AAVrh.56 (See SEQ ID NO: 54 in | AAVrh.56 (See SEQ ID NO: 152 in |
| US20150315612) | US20150315612) |
| AAVrh.57 (See SEQ ID NO: in 497 | AAVrh.57 (See SEQ ID NO: 105 in |
| US20150315612 SEQ ID NO: 26 | US20150315612) |
| AAVrh.58 (See SEQ ID NO: 27 in | AAVrh.58 (See SEQ ID NO: 48 in |
| US20150315612) | US20150159173 and SEQ ID NO: 106 in |
| | US20150315612) |
| | AAVrh.58 (See SEQ ID NO: 232 in |
| | US20150315612) |
| AAVrh.59 (See SEQ ID NO: 42 in | AAVrh.59 (See SEQ ID NO: 110 in |
| US20150315612) | US20150315612) |
| AAVrh.60 (See SEQ ID NO: 31 in | AAVrh.60 (See SEQ ID NO: 120 in |
| US20150315612) | US20150315612) |
| AAVrh.61 (See SEQ ID NO: 107 in | AAVrh.61 (AAV2-3) (See SEQ ID NO: 21 in |
| US20150315612) | US20150315612) |
| AAVrh.62 (AAV2-15) (See SEQ ID NO: 33 in | AAVrh.62 (AAV2-15) (See SEQ ID NO: 114 |
| US20150315612) | in US20150315612) |
| AAVrh.64 (See SEQ ID NO: 15 in | AAVrh.64 (See SEQ ID NO: 43 in |
| US20150315612) | US20150159173 and SEQ ID NO: 99 in |
| | US20150315612) |
| | AAVrh.64 (See SEQ ID NO: 233 in |
| | US20150315612) |
| AAVRh.64Rl (See SEQ ID NO: in | AAVRh.64R2 (See SEQ ID NO: in |
| US20150159173 | US20150159173 |
| AAVrh.65 (See SEQ ID NO: 35 in | AAVrh.65 (See SEQ ID NO: 112 in |
| US20150315612) | US20150315612) |
| AAVrh.67 (See SEQ ID NO: 36 in | AAVrh.67 (See SEQ ID NO: 230 in |
| US20150315612) | US20150315612) |
| AAVrh.67 (See SEQ ID NO: 47 in | |
| US20150159173 and SEQ ID NO: 47 in | |
| US20150315612) | |
| AAVrh.68 (See SEQ ID NO: 16 in | AAVrh.68 (See SEQ ID NO: 100 in |
| US20150315612) | US20150315612) |
| AAVrh.69 (See SEQ ID NO: 39 in | AAVrh.69 (See SEQ ID NO: 119 in |
| US20150315612) | US20150315612) |
| AAVrh.70 (See SEQ ID NO: 20 in | AAVrh.70 (See SEQ ID NO: 98 in |
| US20150315612) | US20150315612) |
| AAVrh.71 (See SEQ ID NO: 162 in | AAVrh.72 (See SEQ ID NO: 9 in |
| US20150315612) | US20150315612) |
| AAVrh.73 (See SEQ ID NO: 5 in | AAVrh.74 (See SEQ ID NO: 6 in |
| US20150159173) | US20150159173) |
| AAVrh.8 (See SEQ ID NO: 41 in | AAVrh.8 (See SEQ ID NO: 235 in |
| US20150159173) | US20150315612) |
| AAVrh.8R (See SEQ ID NO: 9 in | AAVrh.8R A586R mutant (See SEQ ID NO: 10 |
| US20150159173, WO2015168666) | in WO2015168666) |
| AAVrh.8R R533A mutant (See SEQ ID NO: 11 in | BAAV (bovine AAV) (See SEQ ID NO: 8 in |
| WO2015168666) | US9193769) |
| BAAV (bovine AAV) (See SEQ ID NO: 10 in | BAAV (bovine AAV) (See SEQ ID NO: 4 in |
| US9193769) | US9193769) |
| BAAV (bovine AAV) (See SEQ ID NO: 2 in | BAAV (bovine AAV) (See SEQ ID NO: 6 in |
| US9193769) | US9193769) |
| BAAV (bovine AAV) (See SEQ ID NO: 1 in | BAAV (bovine AAV) (See SEQ ID NO: 5 in |
| US9193769) | US9193769) |
| BAAV (bovine AAV) (See SEQ ID NO: 3 in | BAAV (bovine AAV) (See SEQ ID NO: 11 in |
| US9193769) | US9193769) |
| BAAV (bovine AAV) (See SEQ ID NO: 5 in | BAAV (bovine AAV) (See SEQ ID NO: 6 in |
| US7427396) | US7427396) |
| BAAV (bovine AAV) (See SEQ ID NO: 7 in | BAAV (bovine AAV) (See SEQ ID NO: 9 in |
| US9193769) | US9193769) |
| BNP61 AAV (See SEQ ID NO: 1 in | BNP61 AAV (See SEQ ID NO: 2 in |
| US20150238550) | US20150238550) |
| BNP62 AAV (See SEQ ID NO: 3 in | BNP63 AAV (See SEQ ID NO: 4 in |
| US20150238550) | US20150238550) |
| caprine AAV (See SEQ ID NO: 3 in US7427396) | caprine AAV (See SEQ ID NO: 4 in |
| | US7427396) |
| true type AAV (ttAAV) (See SEQ ID NO: 2 in | AAAV (Avian AAV) (See SEQ ID NO: 12 in |
| WO2015121501) | US9238800) |
| AAAV (Avian AAV) (See SEQ ID NO: 2 in | AAAV (Avian AAV) (See SEQ ID NO: 6 in |
| US9238800) | US9238800) |
| AAAV (Avian AAV) (See SEQ ID NO: 4 in | AAAV (Avian AAV) (See SEQ ID NO: 8 in |
| US9238800) | US9238800) |
| AAAV (Avian AAV) (See SEQ ID NO: 14 in | AAAV (Avian AAV) (See SEQ ID NO: 10 in |
| US9238800) | US9238800) |
| AAAV (Avian AAV) (See SEQ ID NO: 15 in | AAAV (Avian AAV) (See SEQ ID NO: 5 in |
| US9238800) | US9238800) |
| AAAV (Avian AAV) (See SEQ ID NO: 9 in | AAAV (Avian AAV) (See SEQ ID NO: 3 in |
| US9238800) | US9238800) |
| AAAV (Avian AAV) (See SEQ ID NO: 7 in | AAAV (Avian AAV) (See SEQ ID NO: 11 in |
| US9238800) | US9238800) |
| AAAV (Avian AAV) (See SEQ ID NO: in | AAAV (Avian AAV) (See SEQ ID NO: 1 in |
| US9238800) | US9238800) |
| AAV Shuffle 100-1 (See SEQ ID NO: 23 in | AAV Shuffle 100-1 (See SEQ ID NO: 11 in |
| US20160017295) | US20160017295) |
| AAV Shuffle 100-2 (See SEQ ID NO: 37 in | AAV Shuffle 100-2 (See SEQ ID NO: 29 in |
| US20160017295) | US20160017295) |
| AAV Shuffle 100-3 (See SEQ ID NO: 24 in | AAV Shuffle 100-3 (See SEQ ID NO: 12 in |
| US20160017295) | US20160017295) |
| AAV Shuffle 100-7 (See SEQ ID NO: 25 in | AAV Shuffle 100-7 (See SEQ ID NO: 13 in |
| US20160017295) | US20160017295) |
| AAV Shuffle 10-2 (See SEQ ID NO: 34 in | AAV Shuffle 10-2 (See SEQ ID NO: 26 in |
| US20160017295) | US20160017295) |
| AAV Shuffle 10-6 (See SEQ ID NO: 35 in | AAV Shuffle 10-6 (See SEQ ID NO: 27 in |
| US20160017295) | US20160017295) |
| AAV Shuffle 10-8 (See SEQ ID NO: 36 in | AAV Shuffle 10-8 (See SEQ ID NO: 28 in |
| US20160017295) | US20160017295) |
| AAV SM 100-10 (See SEQ ID NO: 41 in | AAV SM 100-10 (See SEQ ID NO: 33 in |
| US20160017295) | US20160017295) |
| AAV SM 100-3 (See SEQ ID NO: 40 in | AAV SM 100-3 (See SEQ ID NO: 32 in |
| US20160017295) | US20160017295) |
| AAV SM 10-1 (See SEQ ID NO: 38 in | AAV SM 10-1 (See SEQ ID NO: 30 in |
| US20160017295) | US20160017295) |
| AAV SM 10-2 (See SEQ ID NO: 10 in | AAV SM 10-2 (See SEQ ID NO: 22 in |
| US20160017295) | US20160017295) |
| AAV SM 10-8 (See SEQ ID NO: 39 in | AAV SM 10-8 (See SEQ ID NO: 31 in |
| US20160017295) | US20160017295) |
| AAV CBr-7.1 (See SEQ ID NO: 4 in | AAV CBr-7.1 (See SEQ ID NO: 54 in |
| WO2016065001) | WO2016065001) |
| AAV CBr-7.10 (See SEQ ID NO: 11 in | AAV CBr-7.10 (See SEQ ID NO: 61 in |
| WO2016065001) | WO2016065001) |
| AAV CBr-7.2 (See SEQ ID NO: 5 in | AAV CBr-7.2 (See SEQ ID NO: 55 in |
| WO2016065001) | WO2016065001) |
| AAV CBr-7.3 (See SEQ ID NO: 6 in | AAV CBr-7.3 (See SEQ ID NO: 56 in |
| WO2016065001) | WO2016065001) |
| AAV CBr-7.4 (See SEQ ID NO: 7 in | AAV CBr-7.4 (See SEQ ID NO: 57 in |
| WO2016065001) | WO2016065001) |
| AAV CBr-7.5 (See SEQ ID NO: 8 in | AAV CHt-6.6 (See SEQ ID NO: 35 in |
| WO2016065001) | WO2016065001) |
| AAV CHt-6.6 (See SEQ ID NO: 85 in | AAV CHt-6.7 (See SEQ ID NO: 36 in |
| WO2016065001) | WO2016065001) |
| AAV CHt-6.7 (See SEQ ID NO: 86 in | AAV CHt-6.8 (See SEQ ID NO: 37 in |
| WO2016065001) | WO2016065001) |
| AAV CHt-6.8 (See SEQ ID NO: 87 in | AAV CHt-Pl (See SEQ ID NO: 29 in |
| WO2016065001) | WO2016065001) |
| AAV CHt-Pl (See SEQ ID NO: 79 in | AAV CHt-P2 (See SEQ ID NO: 1 in |
| WO2016065001) | WO2016065001) |
| AAV CHt-P2 (See SEQ ID NO: 51 in | AAV CHt-P5 (See SEQ ID NO: 2 in |
| WO2016065001) | WO2016065001) |
| AAV CHt-P5 (See SEQ ID NO: 52 in | AAV CHt-P6 (See SEQ ID NO: 30 in |
| WO2016065001) | WO2016065001) |
| AAV CHt-P6 (See SEQ ID NO: 80 in | AAV CHt-P8 (See SEQ ID NO: 31 in |
| WO2016065001) | WO2016065001) |
| AAV CHt-P8 (See SEQ ID NO: 81 in | AAV CHt-P9 (See SEQ ID NO: 3 in |
| WO2016065001) | WO2016065001) |
| AAV CHt-P9 (See SEQ ID NO: 53 in | AAV CKd-1 (See SEQ ID NO: 57 in |
| WO2016065001) | US8734809) |
| AAV CKd-1 (See SEQ ID NO: 131 in | AAV CKd-10 (See SEQ ID NO: 58 in |
| US8734809) | US8734809) |
| AAV CKd-10 (See SEQ ID NO: 132 in | AAV CKd-2 (See SEQ ID NO: 59 in |
| US8734809) | US8734809) |
| AAV CKd-2 (See SEQ ID NO: 133 in | AAV CKd-3 (See SEQ ID NO: 60 in |
| US8734809) | US8734809) |
| AAV CKd-3 (See SEQ ID NO: 134 in | AAV CKd-4 (See SEQ ID NO: 61 in |
| US8734809) | US8734809) |
| AAV CKd-4 (See SEQ ID NO: 135 in | AAV CKd-6 (See SEQ ID NO: 62 in |
| US8734809) | US8734809) |
| AAV CKd-6 (See SEQ ID NO: 136 in | AAV CKd-7 (See SEQ ID NO: 63 in |
| US8734809) | US8734809) |
| AAV CKd-7 (See SEQ ID NO: 137 in | AAV CKd-8 (See SEQ ID NO: 64 in |
| US8734809) | US8734809) |
| AAV CKd-8 (See SEQ ID NO: 138 in | AAV CKd-B 1 (See SEQ ID NO: 73 in |
| US8734809) | US8734809) |
| AAV CKd-B 1 (See SEQ ID NO: 147 in | AAV CKd-B2 (See SEQ ID NO: 74 in |
| US8734809) | US8734809) |
| AAV CKd-B2 (See SEQ ID NO: 148 in | AAV CKd-B3 (See SEQ ID NO: 75 in |
| US8734809) | US8734809) |
| AAV CKd-B3 (See SEQ ID NO: in US8734809 | AAV CKd-B3 (See SEQ ID NO: 149 in |
| | US8734809) |
| AAV CLv-1 (See SEQ ID NO: 65 in US8734809) | AAV CLv-1 (See SEQ ID NO: 139 in |
| | US8734809) |
| AAV CLvl-1 (See SEQ ID NO: 171 in | AAV Civ 1-10 (See SEQ ID NO: 178 in |
| US8734809) | US8734809) |
| AAV CLvl-2 (See SEQ ID NO: 172 in | AAV CLv-12 (See SEQ ID NO: 66 in |
| US8734809) | US8734809) |
| AAV CLv-12 (See SEQ ID NO: 140 in | AAV CLvl-3 (See SEQ ID NO: 173 in |
| US8734809) | US8734809) |
| AAV CLv-13 (See SEQ ID NO: 67 in | AAV CLv-13 (See SEQ ID NO: 141 in |
| US8734809) | US8734809) |
| AAV CLvl-4 (See SEQ ID NO: 174 in | AAV Civ 1-7 (See SEQ ID NO: 175 in |
| US8734809) | US8734809) |
| AAV Civ 1-8 (See SEQ ID NO: 176 in | AAV Civ 1-9 (See SEQ ID NO: 177 in |
| US8734809) | US8734809) |
| AAV CLv-2 (See SEQ ID NO: 68 in US8734809) | AAV CLv-2 (See SEQ ID NO: 142 in |
| | US8734809) |
| AAV CLv-3 (See SEQ ID NO: 69 in US8734809) | AAV CLv-3 (See SEQ ID NO: 143 in |
| | US8734809) |
| AAV CLv-4 (See SEQ ID NO: 70 in US8734809) | AAV CLv-4 (See SEQ ID NO: 144 in |
| | US8734809) |
| AAV CLv-6 (See SEQ ID NO: 71 in US8734809) | AAV CLv-6 (See SEQ ID NO: 145 in |
| | US8734809) |
| AAV CLv-8 (See SEQ ID NO: 72 in US8734809) | AAV CLv-8 (See SEQ ID NO: 146 in |
| | US8734809) |
| AAV CLv-Dl (See SEQ ID NO: 22 in | AAV CLv-Dl (See SEQ ID NO: 96 in |
| US8734809) | US8734809) |
| AAV CLv-D2 (See SEQ ID NO: 23 in | AAV CLv-D2 (See SEQ ID NO: 97 in |
| US8734809) | US8734809) |
| AAV CLv-D3 (See SEQ ID NO: 24 in | AAV CLv-D3 (See SEQ ID NO: 98 in |
| US8734809) | US8734809) |
| AAV CLv-D4 (See SEQ ID NO: 25 in | AAV CLv-D4 (See SEQ ID NO: 99 in |
| US8734809) | US8734809) |
| AAV CLv-D5 (See SEQ ID NO: 26 in | AAV CLv-D5 (See SEQ ID NO: 100 in |
| US8734809) | US8734809) |
| AAV CLv-D6 (See SEQ ID NO: 27 in | AAV CLv-D6 (See SEQ ID NO: 101 in |
| US8734809) | US8734809) |
| AAV CLv-D7 (See SEQ ID NO: 28 in | AAV CLv-D7 (See SEQ ID NO: 102 in |
| US8734809) | US8734809) |
| AAV CLv-D8 (See SEQ ID NO: 29 in | AAV CLv-D8 (See SEQ ID NO: 103 in |
| US8734809) | US8734809); AAV CLv-Kl 762, see SEQ ID |
| | NO: 18 in WO2016065001) |
| AAV CLv-Kl (See SEQ ID NO: 68 in | AAV CLv-K3 (See SEQ ID NO: 19 in |
| WO2016065001) | WO2016065001) |
| AAV CLv-K3 (See SEQ ID NO: 69 in | AAV CLv-K6 (See SEQ ID NO: 20 in |
| WO2016065001) | WO2016065001) |
| AAV CLv-K6 (See SEQ ID NO: 70 in | AAV CLv-L4 (See SEQ ID NO: 15 in |
| WO2016065001) | WO2016065001) |
| AAV CLv-L4 (See SEQ ID NO: 65 in | AAV CLv-L5 (See SEQ ID NO: 16 in |
| WO2016065001) | WO2016065001) |
| AAV CLv-L5 (See SEQ ID NO: 66 in | AAV CLv-L6 (See SEQ ID NO: 17 in |
| WO2016065001) | WO2016065001) |
| AAV CLv-L6 (See SEQ ID NO: 67 in | AAV CLv-Ml (See SEQ ID NO: 21 in |
| WO2016065001) | WO2016065001) |
| AAV CLv-Ml (See SEQ ID NO: 71 in | AAV CLv-Mll (See SEQ ID NO: 22 in |
| WO2016065001) | WO2016065001) |
| AAV CLv-Ml 1 (See SEQ ID NO: 72 in | AAV CLv-M2 (See SEQ ID NO: 23 in |
| WO2016065001) | WO2016065001) |
| AAV CLv-M2 (See SEQ ID NO: 73 in | AAV CLv-M5 (See SEQ ID NO: 24 in |
| WO2016065001) | WO2016065001) |
| AAV CLv-M5 (See SEQ ID NO: 74 in | AAV CLv-M6 (See SEQ ID NO: 25 in |
| WO2016065001) | WO2016065001) |
| AAV CLv-M6 (See SEQ ID NO: 75 in | AAV CLv-M7 (See SEQ ID NO: 26 in |
| WO2016065001) | WO2016065001) |
| AAV CLv-M7 (See SEQ ID NO: 76 in | AAV CLv-M8 (See SEQ ID NO: 27 in |
| WO2016065001) | WO2016065001) |
| AAV CLv-M8 (See SEQ ID NO: 77 in | AAV CLv-M9 (See SEQ ID NO: 28 in |
| WO2016065001) | WO2016065001) |
| AAV CLv-M9 (See SEQ ID NO: 78 in | AAV CLv-Rl (See SEQ ID NO: 30 in |
| WO2016065001) | US8734809) |
| AAV CLv-Rl (See SEQ ID NO: 104 in | AAV CLv-R2 (See SEQ ID NO: 31 in |
| US8734809) | US8734809) |
| AAV CLv-R2 (See SEQ ID NO: 105 in | AAV CLv-R3 (See SEQ ID NO: 32 in |
| US8734809) | US8734809) |
| AAV CLv-R3 (See SEQ ID NO: 106 in | AAV CLv-R4 (See SEQ ID NO: 33 in |
| US8734809) | US8734809) |
| AAV CLv-R4 (See SEQ ID NO: 107 in | AAV CLv-R5 (See SEQ ID NO: 34 in |
| US8734809) | US8734809) |
| AAV CLv-R5 (See SEQ ID NO: 108 in | AAV CLv-R6 (See SEQ ID NO: 35 in |
| US8734809) | US8734809) |
| AAV CLv-R6 (See SEQ ID NO: 109 in | AAV CLv-R7 (See SEQ ID NO: 110 in |
| US8734809); AAV CLv-R7 802 (see SEQ ID NO: | US8734809) |
| 36 in US8734809) | |
| AAV CLv-R8 (See SEQ ID NO: 37 in | AAV CLv-R8 (See SEQ ID NO: 111 in |
| US8734809) | US8734809) |
| AAV CLv-R9 (See SEQ ID NO: 38 in | AAV CLv-R9 (See SEQ ID NO: 112 in |
| US8734809) | US8734809) |
| AAV CSp-1 (See SEQ ID NO: 45 in US8734809) | AAV CSp-1 (See SEQ ID NO: 119 in |
| | US8734809) |
| AAV CSp-10 (See SEQ ID NO: 46 in US8734809) | AAV CSp-10 (See SEQ ID NO: 120 in |
| | US8734809) |
| AAV CSp-11 (See SEQ ID NO: 47 in US8734809) | AAV CSp-11 (See SEQ ID NO: 121 in |
| | US8734809) |
| AAV CSp-2 (See SEQ ID NO: 48 in US8734809) | AAV CSp-2 (See SEQ ID NO: 122 in |
| AAV CSp-3 (See SEQ ID NO: 49 in US8734809) | US8734809) |
| | AAV CSp-3 (See SEQ ID NO: 123 in |
| | US8734809) |
| AAV CSp-4 (See SEQ ID NO: 50 in US8734809) | AAV CSp-4 (See SEQ ID NO: 124 in |
| | US8734809) |
| AAV CSp-6 (See SEQ ID NO: 51 in US8734809) | AAV CSp-6 (See SEQ ID NO: 125 in |
| | US8734809) |
| AAV CSp-7 (See SEQ ID NO: 52 in US8734809) | AAV CSp-7 (See SEQ ID NO: 126 in |
| | US8734809) |
| AAV CSp-8 (See SEQ ID NO: 53 in US8734809) | AAV CSp-8 (See SEQ ID NO: 127 in |
| | US8734809) |
| AAV CSp-8.10 (See SEQ ID NO: 38 in | AAV CSp-8.10 (See SEQ ID NO: 88 in |
| WO2016065001) | WO2016065001) |
| AAV CSp-8.2 (See SEQ ID NO: 39 in | AAV CSp-8.2 (See SEQ ID NO: 89 in |
| WO2016065001) | WO2016065001) |
| AAV CSp-8.4 (See SEQ ID NO: 40 in | AAV CSp-8.4 (See SEQ ID NO: 90 in |
| WO2016065001) | WO2016065001) |
| AAV CSp-8.5 (See SEQ ID NO: 41 in | AAV CSp-8.5 (See SEQ ID NO: 91 in |
| WO2016065001) | WO2016065001) |
| AAV CSp-8.6 (See SEQ ID NO: 42 in | AAV CSp-8.6 (See SEQ ID NO: 92 in |
| WO2016065001) | WO2016065001) |
| AAV CSp-8.7 (See SEQ ID NO: 43 in | AAV CSp-8.7 (See SEQ ID NO: 93 in |
| WO2016065001) | WO2016065001) |
| AAV CSp-8.8 (See SEQ ID NO: 44 in | AAV CSp-8.8 (See SEQ ID NO: 94 in |
| WO2016065001) | WO2016065001) |
| AAV CSp-8.9 (See SEQ ID NO: 45 in | AAV CSp-8.9 (See SEQ ID NO: 95 in |
| WO2016065001) | WO2016065001) |
| AAV CSp-9 842 (See SEQ ID NO: 54 in | AAV CSp-9 (See SEQ ID NO: 128 in |
| US8734809) | US8734809) |
| AAV.hu.48R3 (See SEQ ID NO: 183 in | AAV.VR-355 (See SEQ ID NO: 181 in |
| US8734809) | US8734809) |
| AAV3B (See SEQ ID NO: 48 in WO2016065001) | AAV3B (See SEQ ID NO: 98 in WO2016065001) |
| AAV4 (See SEQ ID NO: 49 in WO2016065001) | AAV4 (See SEQ ID NO: 99 in WO2016065001) |
| AAV5 (See SEQ ID NO: 50 in WO2016065001) | AAV5 (See SEQ ID NO: 100 in WO2016065001) |
| AAVF1/HSC1 (See SEQ ID NO: 20 in | AAVF1/HSC1 (See SEQ ID NO: 2 in |
| WO2016049230) | WO2016049230) |
| AAVF11/HSC11 (See SEQ ID NO: 26 in | AAVF11/HSC11 (See SEQ ID NO: 4 in |
| WO2016049230) | WO2016049230) |
| AAVF12/HSC12 (See SEQ ID NO: 30 in | AAVF12/HSC12 (See SEQ ID NO: 12 in |
| WO2016049230) | WO2016049230) |
| AAVF13/HSC13 (See SEQ ID NO: 31 in | AAVF13/HSC13 (See SEQ ID NO: 14 in |
| WO2016049230) | WO2016049230) |
| AAVF14/HSC14 (See SEQ ID NO: 32 in | AAVF14/HSC14 (See SEQ ID NO: 15 in |
| WO2016049230) | WO2016049230) |
| AAVF15/HSC15 (See SEQ ID NO: 33 in | AAVF15/HSC15 (See SEQ ID NO: 16 in |
| WO2016049230) | WO2016049230) |
| AAVF16/HSC16 (See SEQ ID NO: 34 in | AAVF16/HSC16 (See SEQ ID NO: 17 in |
| WO2016049230) | WO2016049230) |
| AAVF17/HSC17 (See SEQ ID NO: 35 in | AAVF17/HSC17 (See SEQ ID NO: 13 in |
| WO2016049230) | WO2016049230) |
| AAVF2/HSC2 (See SEQ ID NO: 21 in | AAVF2/HSC2 (See SEQ ID NO: 3 in |
| WO2016049230) | WO2016049230) |
| AAVF3/HSC3 (See SEQ ID NO: 22 in | AAVF3/HSC3 (See SEQ ID NO: 5 in |
| WO2016049230) | WO2016049230) |
| AAVF4/HSC4 (See SEQ ID NO: 23 in | AAVF4/HSC4 (See SEQ ID NO: 6 in |
| WO2016049230) | WO2016049230) |
| AAVF5/HSC5 (See SEQ ID NO: 25 in | AAVF5/HSC5 (See SEQ ID NO: 11 in |
| WO2016049230) | WO2016049230) |
| AAVF6/HSC6 (See SEQ ID NO: 24 in | AAVF6/HSC6 (See SEQ ID NO: 7 in |
| WO2016049230) | WO2016049230) |
| AAVF7/HSC7 (See SEQ ID NO: 27 in | AAVF7/HSC7 (See SEQ ID NO: 8 in |
| WO2016049230) | WO2016049230) |
| AAVF8/HSC8 (See SEQ ID NO: 28 in | AAVF8/HSC8 (See SEQ ID NO: 9 in |
| WO2016049230) | WO2016049230) |
| AAVF9/HSC9 (See SEQ ID NO: 10 in | AAVF9/HSC9 882 (see SEQ ID NO: 29 in |
| WO2016049230) | WO2016049230) |

Table 4 below describe exemplary chimeric or variant capsid proteins that can be used as the AAV capsid in the rAAV vector described herein, or with any combination with wild type capsid proteins and/or other chimeric or variant capsid proteins now known or later identified and each is incorporated herein. In some embodiments, the rAAV vector encompassed for use is a chimeric vector, e.g., as disclosed in U.S. Pat. Nos. 9,012,224 and 7,892,809, which are incorporated herein in their entirety by reference.

In some embodiments, the rAAV vector is a haploid rAAV vector, as disclosed in PCT/US18/22725, or polyploid rAAV vector, e.g., as disclosed in PCT/US2018/044632 filed on Jul. 31, 2018 and in U.S. application Ser. No. 16/151,110, each of which are incorporated herein in their entirety by reference. In some embodiments, the rAAV vector is a rAAV3 vector, as disclosed in U.S. Pat. No. 9,012,224 and WO 2017/106236 which are incorporated herein in their entirety by reference. In some embodiments, the rAAV particles are the exemplary rAAVs presented in U.S. Patent Application No. US2018/0371496A1; International Patent Application No. WO2018/170310A1; or U.S. Pat. Nos. 7,892,809; 6,491,907; or 7,172,893, which are incorporated herein by reference in their entireties.

TABLE 4
Exemplary chimeric and rAAV variant capsids

| Chimeric or | | Chimeric or | |
|---|---|---|---|
| variant capsid | reference | variant capsid | reference |
| LK03 and others | Lisowski et al. [REF 1] | AAV-leukemia | Michelfelder S [REF |
| LK0-19 | | targeting | 30] |
| AAV-DJ | Grimm et al., [REF 2] | AAV-tumor targeting | Muller O J, et al., |
| | | | [REF 31] |
| Olig001 | Powell S K et al., [REF 3] | AAV-tumor targeting | Grifman M et al., |
| | | | [REF 32] |
| rAAV2-retro | Tervo D et al., [REF 4] | AAV2 efficient | Girod et al., [REF 33] |
| | | targeting | |
| AAV-LiC | Marsic D et al., [REF 5] | AAVpo2.1, -po4, -poS, | Bello A, et al., [REF |
| | | and -po6). | 34] |
| (AAV-Keral, AAV- | Sallach et al., [REF 6] | AAV rh and AAV Hu | Gao G, et al., [REF |
| Kera2, and AAV- | | | 35] |
| Kera3) | | | |
| AAV 7m8 | Dalkara et al., [REF 7] | AAV-Go.1 | Arbetman A E et al., |
| | | | [REF 36] |
| (AAV1.9 | Asuri P et al., [REF 8] | AAV-mo.1 | Lochrie M A et al., |
| | | | [REF 37] |
| AAV r3.45 | Jang J H et al., [REF 9] | BAAV | Schmidt M, et al., |
| | | | [REF 38] |
| AAV clone 32 and | Gray S J, et al., [REF 10] | AAAV | Bossis I et al., [REF |
| 83) | | | 39] |
| AAV-U87R7-C5 | Maguire et al., [REF 11] | AAV variants | Chen C L et al., [REF |
| | | | 40] |
| AAV ShH13, AAV | Koerberetal., [REF 12] | AAV8 K137R | Sen D et al., [REF 41] |
| ShH19, AAV Ll-12 | | | |
| AAV HAE-1, AAV | Li W et al., [REF 13] | AAV2 Y | Li B, et al., [REF 42] |
| HAE-2 | | | |
| AAV variant ShH10 | Klimczak et al., [REF 14] | AAV2 | Gabriel N et al., [REF |
| | | | 43] |
| AAV2.5T | Excoffon et al., [REF 15] | AAV Anc80L65 | Zinn E, et al., [REF |
| | | | 44] |
| AAV LS1-4, AAV | Sellner L et al., [REF 16] | AAV2G9 | Shen S et al., [REF |
| Lsm | | | 45] |
| AAV1289 | Li W, et al., [REF 17] | AAV2 265 insertion- | Li C, et al., [REF 46] |
| | | AAV2/265D | |
| AAVHSC 1-17 | Charbel Issa P et al., | AAV2.5 | Bowles D E, et al., |
| | [REF 18] | | [REF 47] |
| AAV2 Rec 1-4 | Huang W. et al., [REF | AAV3 SASTG | Messina E L et al., |
| | 19] | | [REF 48] and [REF |
| | | | 55], (Piacentio et al., |
| | | | (Hum Gen Ther, |
| | | | 2012, 23: 635-646)) |
| AAV8BP2 | Cronin T, et al., [REF 20] | AAV2i8 | Asokan A et al., [REF |
| | | | 49] |
| AAV-B1 | Choudhury S R, et al., | AAV8G9 | Vance M, et al., [REF |
| | [REF 21] | | 50] |
| AAV-PHP.B | Deverman B E, et al., | AAV2 tyrosine | Zhong L et al., [REF |
| | [REF 22] | mutants AAV2 Y-F | 51] |
| AAV9.45, | Pulicherla N [REF 23], et | AAV8 Y-F and AAV9 | Petrs-Silva H et al., |
| AAV9.61, | al., | Y-F | [REF 52] |
| AAV9.47 | | | |
| AAVM41 | Yang L et al., [REF 24] | AAV6 Y-F | Qiao C et al., [REF |
| | | | 53] |
| AAV2 displayed | Korbelin J et al. [REF | (AAV6.2) PCT | Carlon M, et al., [REF |
| peptides) | 25], | Publication No. | 54] |
| | | WO2013158879Al | |
| | | (lysine mutants) | |
| AAV2-GMN | Geoghegan J C [REF 26] | | |
| AAV9-peptide | Varadi K, et al., [REF 27] | | |
| displayed | | | |
| AAV8 and AAV9 | Michelfelder et al., [REF | | |
| peptide displayed | 28] | | |
| AAV2-muscle | YuCY et al., [REF 29] | | |
| targeting peptide | | | |

In one embodiment, the viral vector as disclosed herein comprises a capsid protein, associated with any of the following biological sequence files listed in the file wrappers of USPTO issued patents and published applications, which describe chimeric or variant capsid proteins that can be incorporated into the AAV capsid of this invention in any combination with wild type capsid proteins and/or other chimeric or variant capsid proteins now known or later identified (for demonstrative purposes, 11486254 corresponds to U.S. patent application Ser. No. 11/486,254 and the other biological sequence files are to be read in a similar manner): 11486254.raw, 11932017.raw, 12172121.raw, 12302206.raw, 12308959.raw, 12679144.raw, 13036343.raw, 13121532.raw, 13172915.raw, 13583920.raw, 13668120.raw, 13673351.raw, 13679684.raw, 14006954.raw, 14149953.raw, 14192101.raw, 14194538.raw, 14225821.raw, 14468108.raw, 14516544.raw, 14603469.raw, 14680836.raw, 14695644.raw, 14878703.raw, 14956934.raw, 15191357.raw, 15284164.raw, 15368570.raw, 15371188.raw, 15493744.raw, 15503120.raw, 15660906.raw, and 15675677.raw.

In an embodiment, the AAV capsid proteins and virus capsids of this invention can be chimeric in that they can comprise all or a portion of a capsid subunit from another virus, optionally another parvovirus or AAV, e.g., as described in international patent publication WO 00/28004, which is incorporated by reference.

In some embodiments, an rAAV vector genome is single stranded or a monomeric duplex as described in U.S. Pat. No. 8,784,799, which is incorporated herein.

As a further embodiment, the AAV capsid proteins and virus capsids of this invention can be polyploid (also referred to as haploid) in that they can comprise different combinations of VP1, VP2 and VP3 AAV serotypes in a single AAV capsid as described in PCT/US18/22725, which is incorporated by reference.

In order to facilitate their introduction into a cell, a viral vector genome, e.g., the rAAV vector genome, useful in the invention are recombinant nucleic acid constructs that include (1) a heterologous sequence to be expressed and (2) viral sequence elements that facilitate integration and expression of the heterologous genes. The viral sequence elements may include those sequences of an AAV vector genome that are required in cis for replication and packaging (e.g., functional ITRs) of the DNA into an AAV capsid. In an embodiment, such an rAAV vector genome may also contain marker or reporter genes. In an embodiment, an rAAV vector genome can have one or more of the AAV3b wild-type (WT) cis genes replaced or deleted in whole or in part, but retain functional flanking ITR sequences.

In one embodiment, the viral vector persists (e.g., for an extended period of time) in the target cell (e.g., via replication or recombination, and/or concatemer formation). This may occur through self-replication when the target cell divides and/or concatemer formation, or a combination of both. In embodiments of the invention, the viral vector is converted into a concatemeric structures within a target cell. In embodiments, the concatemeric structures persist (e.g., for an extended period of time) in the target cell (e.g., via replication or recombination). Persistence of the concatemeric structure may be extra-chromosomal (e.g., as a mini-chromosome) or by integration into the target cell chromosome.

The methods described herein promote sustained expression of a predetermined nucleic acid, e.g., the heterologous nucleic acid, in a target cell or population thereof. By sustained expression is meant that the expression of encoded product, e.g., protein or nucleic acid, is at a detectable level that persists for an extended period of time, if not indefinitely, following administration of the subject vector. By extended period of time is meant from 1-5 weeks, from 2-5 weeks, from 3-5 weeks, from 4-5 weeks, at least 5 weeks, at least 6 weeks, at least 1 month, at least 2 months, at least 3 months, at least 4 months, at least 5 months, at least 6 months, at least 7, 8, 9, 10, 11 or 12 months, from 1-12 months, from 1-10 years, at least 1, 2, 3, 4, 5, 6, 7, 8, 9, or 10 years, or longer. By detectable level is meant that the expression of the encoded product is at a level such that one can detect the encoded product in target cell, or the mammal comprising the same, e.g., in the serum of the mammal, at a therapeutic concentration. As compared to a control in which the pBluescript plasmid vector (Stratagene Corporation, La Jolla, Calif.) is employed, protein expression persists for a period of time at a detectable level that is at least about 2 fold, usually at least about 5 fold and more usually at least about 10 fold longer following the subject methods as compared to a control. An encoded product is considered to be at a detectable level if it can be detected using technology and protocols readily available and well known to those of skill in the art.

In some embodiments, the above-described sustained expression is not only at a detectable level, but at a high level. A minimal vector is considered to provide for a high level of expression if, after a period of time following its administration, e.g., at least about 28 days, the protein or nucleic acid encoded by the expression cassette of the vector is present at high levels in the host, e.g., in the target cells, in the serum of the host, etc. Levels of an encoded product are considered “high” for purposes of the invention if they are present in amounts such that they exhibit detectable activity (e.g., have an impact on the phenotype), e.g., therapeutic activity, in the host. Whether or not the expression level of a particular product is high will necessarily vary depending on the nature of the particular product, but can readily be determined by those of skill in the art, e.g., by an evaluation of whether expression of the product is sufficient to exhibit a desired effect on the phenotype of the host, such as an amelioration of a disease symptom.

In one embodiment, co-administration of the viral vector and the antibiotic increases sustained expression in the cell or population thereof as compared to a reference level. As used herein, “reference level” refers to the duration of sustained expression in an otherwise identical sample that is not co-administered an antibiotic, i.e., is only administered the viral vector. In one embodiment, co-administration of the viral vector and the antibiotic increases sustained expression in the cell or population thereof by at least 1-5 weeks, from 2-5 weeks, from 3-5 weeks, from 4-5 weeks, at least 5 weeks, at least 6 weeks, at least 1 month, at least 2 months, at least 3 months, at least 4 months, at least 5 months, at least 6 months, at least 7, 8, 9, 10, 11 or 12 months, from 1-12 months, from 1-10 years, at least 1, 2, 3, 4, 5, 6, 7, 8, 9, or 10 years, or longer, or at least 5%, 10%, 15%, 20%, 25%, 30%, 35%, 40%, 45%, 50%, 55%, 60%, 65%, 70%, 75%, 80%, 85%, 90%, 95%, 99% or more, or at least 10-fold, 20-fold, 30-fold, 40-fold, 50-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 410-fold, 420-fold, 430-fold, 440-fold, 450-fold, 460-fold, 470-fold, 480-fold, 490-fold, 500-fold, 550-fold, 600-fold, 650-fold, 700-fold, 750-fold, 800-fold, 850-fold, 900-fold, 950-fold, 1000-fold as compared to a reference level.

In one embodiment, expression of a viral vector, e.g., the AAV vector, is localized to a specific organ or tissue. Exemplary organs or tissues include, the liver (or specifically the liver right lobe, liver left lobe, liver median lobe, liver caudate lobe), spleen, Brain, Skeletal Muscle, Heart, Aorta, lungs, blood vessels, pancreas, bladder, reproductive system, small intestine, large intestine, esophagus, rectum, thyroid, diaphragm, stomach, kidney, or the like. In one embodiment, expression of the viral vector is localized to at least two organs or tissue types. Methods for detecting expression of a vector are known in the art and include, e.g., microscopy of an isolated organ or tissue, or FACS of cells obtained from an isolated organ or tissue.

### Antibiotics Administration

Methods described herein require co-administration of an antibiotic and a viral vector, e.g., to reduce toxicity associated with administration of a viral vector. In one embodiment, the antibiotic is a member of the tetracycline family of antibiotics, or a member of the macrolides family of antibiotics.

Tetracycline members are broad-spectrum antibiotic compounds that are either isolated directly from several species of Streptomyces bacteria or produced semi-synthetically from those isolated compounds. Tetracycline molecules comprise a linear fused tetracyclic nucleus (rings designated A, B, C and D) to which a variety of functional groups are attached. Tetracyclines are named for their four (“tetra-”) hydrocarbon rings (“-cycl-”) derivation (“-me”). They are defined as a subclass of polyketides, having an octahydrotetracene-2-carboxamide skeleton and are known as derivatives of polycyclic naphthacene carboxamide. While all tetracyclines families have a common structure, they differ from each other by the presence of chloride, methyl, and hydroxyl groups.

Tetracyclines have been used extensively in prophylaxis and in treatment of human and animal infections, as well as at subtherapeutic levels in animal feed as growth promoters.

Tetracyclines function to inhibit growth of the infectious agent (e.g., a bacteriostatic) via inhibition of protein synthesis by binding reversibly to the bacterial 30S ribosomal subunit and preventing the aminoacyl tRNA from binding to the A site of the ribosome. They also bind to some extent the bacterial 50S ribosomal subunit and may alter the cytoplasmic membrane causing intracellular components to leak from bacterial cells.

Exemplary tetracycline members include Tetracycline, Chlortetracycline, Oxytetracycline, Demeclocycline, Lymecycline, Meclocycline, Methacycline, Minocycline, Rolitetracycline, Doxycycline, Tigecycline, Eravacycline, Sarecycline, and Omadacycline.

Various tetracycline members are further described in U.S. Pat. Nos. 3,957,980; 3,674,859; 2,980,584; 2,990,331; 3,062,717; 3,557,280; 4,018,889; 4,024,272; 4,126,680; 3,454,697; and 3,165,531, the contents of which are incorporated herein by reference in its entirety.

Macrolide member are named for their characteristic 14- to 16-membered ring. The macrolides also often have one or more 6-membered sugar-derived rings attached to the main macrolide ring. The first macrolide member to be developed was erythromycin, which was isolated from a soil sample from the Philippines in 1952. Even though erythromycin has been one of the most widely prescribed antibiotics, its disadvantages are relatively low bioavailability, gastrointestinal side effects, and a limited spectrum of activity. Another exemplary macrolide is the compound, azithromycin, which is an azolide derivative of erythromycin incorporating a methyl-substituted nitrogen in the macrolide ring. Azithromycin is sold under the trade name Zithromax®. A more recently introduced macrolide is telithromycin, which is sold under the trade name Ketek®. Telithromycin is a semisynthetic macrolide in which a hydroxyl group of the macrolide ring has been oxidized to a ketone group. See Yong-Ji Wu, Highlights of Semi-Synthetic Developments from Erythromycin A, Current Pharm. Design, vol. 6, pp. 181-223 (2000); Yong-Ji Wu and Wei-uo Su, Recent Developments on Ketolides and Macrolides, Curr. Med. Chem., vol. 8, no. 14, pp. 1727-1758 (2001); and Pal, Sarbani, “A Journey Across the Sequential Development of Macrolides and Ketolides Related to Erythromycin, Tetrahedron 62 (2006) 3171-3200, the contents of which are incorporated herein by reference in their entireties. Additional known macrolide members include Clarithromycin and Fidoximycin.

Further, macrolides have been shown to have an immunomodulatory effect, for example, Azithromycin has been shown to suppress Th1- and Th2-related chemokines upon simulation (see e.g., Kuo, C-H, et al. Journal of Microbiology, Immunology, and Infection (2019) 52, 872-879), and clarithromycin has been shown to modulate IL-8 levels and Neutophil accumulation and activation in refractory asthma patients (see e.g., Simpson, J. L., et al. AM J Respir Crit Care Med (2008) 177, 148-155). The immunomodulatory effect of macrolides is further described in, e.g., Zimmerman, P., et al. Front in Immunol. (2008) 9.302. The contents of these citations are incorporated herein by reference in their entireties.

Azithromycin is further described in U.S. Pat. Nos. 4,474,768 and 4,517,359. Additional macrolides are referred to in U.S. Pat. Nos. 6,159,945; 6,291,656; 6,025,350; 6,407,074; 6,420,536; 6,472,371, and 6,043,227, and European Patent Publication number EP1044208, the contents of which are incorporated herein by reference in its entirety.

Alternatively, administration of the antibiotic can be continuous, either periodic or pulsed. In one embodiment, the antibiotic is administered to the subject at least once. Alternatively, administration of the antibiotic can be periodic or continuous, e.g., over an extended period of time. For example, via a continuous intravenous administration. In one embodiment, the antibiotic is administered to the subject at least twice. For example, a subject can be administered an antibiotic at least 1, 2, 3, 4, 5, times per hour, at least 1, 2, 3, 4, 5, 7, 8, 9, 10, or more times per day, at least 1, 2, 3, 4, 5, 7, 8, 9, 10, or more times per week, or at least 2, 3, 4, 5, 7, 8, 9, 10, or more times per month. In one embodiment, the antibiotic is administered at least once daily for at least 10, 11, 12, 13, 14, 15, 16, 17, 18, 19, 20, 21, 22, 23, 24, 25, 26, 27, 28, 29, 30, or more weeks. In one embodiment, when the antibiotic is administered more than once, the dosage of the antibiotic is the same for each administration, e.g., the antibiotic is administered at 1 gram/day for each administration. In one embodiment, when the antibiotic is administered more than once, the dosage of the antibiotic is different for at least two administrations, e.g., the antibiotic is administered at 2 gram/day for the first administration, and at 1 gram/day for each subsequent administration.

A subject can be “pre-treated with an antibiotic”, meaning they are administered an antibiotic at least once prior to administration of the viral vector. For example, at least one month, week, day, hour, min prior to administration of the viral vector.

In one embodiment, the antibiotic is administered systemically. Antibiotic can be administered using standard routes of administration, e.g., oral, intravenous, or intramuscular administration of the antibiotic. Alternatively, the antibiotic is administered locally to the site of action for the viral vector. For example, when co-administering an antibiotic with a viral vector having liver-specific expression, the antibiotic would be administered directly to the liver. The mode and route of administration of the antibiotic need not be the same as the viral vector. For example, the antibiotic is administered orally for systemic administration and the viral vector is administered locally to the kidney for kidney-specific expression. Alternatively, the mode and route of administration of the antibiotic and viral vector is the same, e.g., the antibiotic and viral vector are administered locally to the kidney for kidney-specific expression.

In certain embodiments, the tetracycline family member of antibiotics is administered in combination with at least one additional antibiotic, e.g., selected from either the tetracycline family members or the macrolides family members of antibiotics. In one embodiment, a combination of antibiotics is administered. The combination of antibiotics include at least one or more antibiotics selected from the group consisting of Gentamicin, Tobramycin, Amikacin, Rifampin, Amphotericin B, Trimethoprim, Sulfamethoxazole, Trimethoprim-sulfamethoxazole, Cephalothin, Chloramphenicol, Clindamycin, Nitrofurantoin, Metronidazole, and Sulfonamides.

In one embodiment, the antibiotic is administered in combination with a blocking agent of immune responses. Exemplary blocking agents include, but is not limited to inhibitory oligodeoxynucleotide (ODN), NF-κB inhibitor (e.g., Bay 11), and complement inhibitor (e.g., decay activating factor (DAF)).

### Compositions

The viral vector and antibiotic described herein can be incorporated into compositions or pharmaceutical compositions suitable for administration to a subject for in vivo delivery to cells, tissues, or organs of the subject. In one embodiment, the composition further comprises a steroid, e.g., prednisone. Alternatively, in one embodiment, the composition does not comprise a steroid, e.g., prednisone.

Typically, the pharmaceutical composition includes the viral vector, e.g., the rAAV vector, and antibiotic described herein and a pharmaceutically acceptable carrier. For example, the viral vector and antibiotic can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high viral vector and antibiotic concentration. Sterile injectable solutions can be prepared by incorporating the viral vector and antibiotic in the required amount in an appropriate buffer with one or a combination of ingredients enumerated above, as required, followed by filtered sterilization. The viral vector and antibiotic can be formulated to deliver a transgene in the nucleic acid, e.g., a therapeutic gene, to the cells of a recipient, resulting in the therapeutic expression of the nucleic acid therein. The composition can also include a pharmaceutically acceptable carrier.

The compositions provided herein can be used to deliver a predetermined DNA sequence (e.g., a therapeutic gene) for various purposes. In some embodiments, the DNA sequence encodes an RNA or protein that is intended to be used for research purposes, e.g., to create a somatic transgenic animal model harboring the transgene, e.g., to study the function of the protein to which the expressed protein or RNA interacts. In another example, the transgene encodes that is intended to be used to create an animal model of disease. In some embodiments, the transgene encodes one or more peptides, polypeptides, or proteins, which are useful for the treatment or prevention of disease states in a mammalian subject.

Pharmaceutical compositions for therapeutic purposes typically must be sterile and stable under the conditions of manufacture and storage. The composition can be formulated as a solution, microemulsion, dispersion, liposomes, or other ordered structure suitable to high viral vector and antibiotic concentration. Sterile injectable solutions can be prepared by incorporating the viral vector and antibiotic in the required amount in an appropriate buffer with one or a combination of ingredients enumerated above, as required, followed by filtered sterilization.

As used herein, “Pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation and is compatible with administration to a subject, for example a human. It must also be suitable for use in contact with any tissues or organs with which it may come in contact, meaning that it must not carry a risk of toxicity, irritation, allergic response, immunogenicity, or any other complication that excessively outweighs its therapeutic benefits. Examples of pharmaceutically acceptable carriers include, but are not limited to, a solvent or dispersing medium containing, for example, water, pH buffered solutions (e.g., phosphate buffered saline (PBS), HEPES, TES, MOPS, etc.), isotonic saline, Ringer's solution, polyol (for example, glycerol, propylene glycol, liquid polyethylene glycol, and the like), alginic acid, ethyl alcohol, and suitable mixtures thereof. In some embodiments, the pharmaceutically acceptable carrier can be a pH buffered solution (e.g. PBS) or water.

### Dosage, Administration and Efficacy

The methods provided herein comprise co-administering to the subject an effective amount of a viral vector and an antibiotic as described herein. As will be appreciated by a skilled practitioner, the term “effective amount” refers to the amount of the viral vector composition administered that results in expression of the encoded protein or RNA in a “therapeutically effective amount” for the treatment of a disease, or the amount of the antibiotic that results in inhibition of toxicity resulting from viral vector administration.

The dosage ranges for the composition comprising a viral vector and/or antibiotic depends upon the potency (e.g., efficiency of the promoter), and includes amounts large enough to produce the desired effect, e.g., expression of the desired protein or RNA, for treatment of a disease, e.g., cancer, and inhibition of toxicity, respectively. The dosage should not be so large as to cause unacceptable adverse side effects. Generally, the dosage will vary with the particular characteristics of the DNA construct, expression efficiency and with the age, condition, and sex of the patient. The dosage can be determined by one of skill in the art. For example, in mice, where a viral vector is intravenously administered in saline, in one embodiment, the amount of viral vector that is administered to a target cell can range from about 1 to 200 ug, or about 10 to 50 ug. One of skill in that adjusts the dose accordingly for administration to humans or larger animals. In certain embodiments the size of the viral vector ranges from about 0.5 to 100 kb, or from about 2 to 15 kb.

In reference to a viral vector, the term “therapeutically effective amount” is an amount of an expressed therapeutic gene product or RNA that is sufficient to produce a statistically significant, measurable change in expression of a disease biomarker or reduction in a given disease symptom (see “Efficacy Measurement” below). Such effective amounts can be gauged in clinical trials as well as animal studies for a given DNA construct or virus particle composition.

In reference to an antibiotic, the term “therapeutically effective amount” is an amount of an expressed therapeutic protein or RNA that is sufficient to produce a statistically significant, measurable change in expression of a disease biomarker or reduction in a given disease symptom (see “Efficacy Measurement” below). Such effective amounts can be gauged in clinical trials as well as animal studies for a given viral vector, e.g., rAAV vector, composition.

“Co-administered,” as used herein, means that two (or more) different treatments (e.g., a viral vector and an antibiotic) are delivered to the subject during the course of the subject's affliction with the disease (e.g., a disease selected from those listed in Table 2), e.g., the two or more treatments are delivered after the subject has been diagnosed with a disease and before the disease has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom (e.g., toxicity resulting from by administration of a viral vector), or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The viral vector, e.g., a rAAV vector, described herein and the antibiotic can be administered simultaneously, in the same or in separate compositions, or sequentially. For sequential administration, the viral vector described herein can be administered first, and the antibiotic can be administered second, or the order of administration can be reversed. The viral vector and/or antibiotic can be administered during periods of active disorder, or during a period of remission or less active disease. The viral vector can be administered before another treatment, concurrently with the treatment, post-treatment, or during remission of the cancer.

When administered in combination, the viral vector and the antibiotic, can be administered in an amount or dose that is higher, lower or the same as the amount or dosage of each agent used individually, e.g., as a monotherapy. In certain embodiments, the administered amount or dosage of the viral vector and the antibiotic is lower or higher (e.g., at least 20%, at least 30%, at least 40%, or at least 50%) than the amount or dosage of each agent used individually, e.g., for a therapeutic effect.

In some embodiments, a single treatment regimen is required for both the viral vector (e.g., rAAV vector) and antibiotic. In other embodiments, administration of one or more subsequent doses or treatment regimens can be performed for both the viral vector and antibiotic. In other embodiments, administration of one or more subsequent doses or treatment regimens can be performed for only one treatment (e.g., the rAAV vector and antibiotic). For example, after treatment biweekly for three months, treatment can be repeated once per month, for six months or a year or longer. In some embodiments, no additional treatments are administered following the initial treatment of one or both of the viral vector and antibiotic.

Parenteral Dosage Forms

Parenteral dosage forms of a composition or agent described herein can be administered to a subject by various routes, including, but not limited to, epidural, intracerebral, intracerebroventricular, epicutaneous, nasal administration, intraarterial, intraarticular, intracardiac, intracavernous injection, intradermal, intralesional, intramuscular, intraocular, intraosseous infusion, intraperitoneal, intrathecal, intrauterine, intravaginal administration, intravenous, intravesical, intravitreal, subcutaneous, transdermal, perivascular administration, or transmucosal. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Agents useful in the methods and compositions described herein can be administered topically, intravenously (by bolus or continuous infusion), intracellular injection, intratissue injection, orally, by inhalation, intraperitoneally, intramuscularly, subcutaneously, intracavity, and can be delivered by peristaltic means, if desired, or by other means known by those skilled in the art. The agent can be administered systemically, if so desired.

Efficacy

The efficacy of a given treatment for a given disease or disorder, such as a disease listed in Table 2, can be determined by the skilled clinician. However, a treatment is considered “effective treatment,” as the term is used herein, if any one or all of the signs or symptoms of the disease or disorder is/are altered in a beneficial manner, or other clinically accepted symptoms or markers of disease are improved, or ameliorated, e.g., by at least 10% following treatment with a DNA construct encoding a therapeutic protein or RNA. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the disease (e.g., cancer); or (2) relieving the disease, e.g., causing regression of symptoms; and (3) preventing or reducing the likelihood of the development of the disease, or preventing secondary diseases/disorders associated with the disease, such as cancer (e.g., cancer metastasis).

An effective amount for the treatment of a disease means that amount which, when administered to a mammal in need thereof, is sufficient to result in effective treatment as that term is defined herein, for that disease. Efficacy of an agent can be determined by assessing physical indicators that are particular to a given disease. For example, physical indicators for cancer include, but are not limited to, pain, tumor size, tumor growth rate, blood cell count, etc.

Dosage

Dosages of the viral vector as disclosed herein to be administered to a subject depend upon the mode of administration, the disease or condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵ transducing units, optionally about 10⁸ to about 10¹³ transducing units.

In one embodiment, the viral vector is administered at a dose of greater than 1e¹². For example, the recombinant viral vector is administered at a dose greater than 1e¹², greater than 1.5e¹², greater than 2e¹², greater than 2.5e¹², greater than 3e¹², greater than 3.5e¹², greater than 4e¹², greater than 4.5e¹², greater than 5e¹², greater than 5.5e¹², greater than 6e¹², greater than 6.5e¹², greater than 7e¹², greater than 7.5e¹², greater than 8e¹², greater than 8.5e¹², greater than 9e¹², greater than 9.5e¹², greater than 1e¹³, greater than 1.5e¹³, greater than 1.6e¹³, greater than 1.8e¹³, greater than 2e¹³, greater than 2.5e¹³, greater than 3e¹³, greater than 3.5e¹³, greater than 4e¹³, greater than 4.5e¹³, greater than 5e¹³, greater than 5.5e¹³, greater than 6e¹³, greater than 6.3e¹³, greater than 6.5e¹³, greater than 6.7e¹³, greater than 7e¹³, greater than 7.5e¹³, greater than 8e¹³, greater than 8.5e¹³, greater than 9e¹³, greater than 9.5e¹³, greater than 1e¹⁴, greater than 1.5e¹⁴, greater than 2e¹⁴, greater than 2.5e¹⁴, greater than 3e¹⁴, greater than 3.5e¹⁴, greater than 4e¹⁴, greater than 4.5e¹⁴, greater than 5e¹⁴, greater than 5.5e¹⁴, greater than 6e¹⁴, greater than 6.5e¹⁴, greater than 7e¹⁴, greater than 7.5e¹⁴, greater than 8e¹⁴, greater than 8.5e¹⁴, greater than 9e¹⁴, greater than 9.5e¹⁴, greater than 1e¹⁵, greater than 1.5e¹⁵, greater than 2e¹⁵, greater than 2.5e¹⁵, greater than 3e¹⁵, greater than 3.5e¹⁵, greater than 4e¹⁵, greater than 4.5e¹⁵, greater than 5e¹⁵, greater than 5.5e¹⁵, greater than 6e¹⁵, greater than 6.5e¹⁵, greater than 7e¹⁵, greater than 7.5e¹⁵, greater than 8e¹⁵, greater than 8.5e¹⁵, greater than 9e¹⁵, greater than 9.5e¹⁵ or, greater than 1e¹⁶.

In the absence of a steroid, e.g., prednisone, or in the presence of a low dose of a steroid, e.g., at least 60 mg/day of prednisone, the recombinant viral vector is administered at a dose that is at least 1e¹², at least 1.5e¹², at least 2e¹², at least 2.5e¹², at least 3e¹², at least 3.5e¹², at least 4e¹², at least 4.5e¹², at least 5e¹², at least 5.5e¹², at least 6e¹², at least 6.5e¹², at least 7e¹², at least 7.5e¹², at least 8e¹², at least 8.5e¹², at least 9e¹², at least 9.5e¹², at least 1e¹³, at least 1.5e¹³, at least 1.6e¹³, at least 1.8e¹³, at least 2e¹³, at least 2.5e¹³, at least 3e¹³, at least 3.5e¹³, at least 4e¹³, at least 4.5e¹³, at least 5e¹³, at least 5.5e¹³, at least 6e¹³, at least 6.3e¹³, at least 6.5e¹³, at least 6.7e¹³, at least 7e¹³, at least 7.5e¹³, at least 8e¹³, at least 8.5e¹³, at least 9e¹³, at least 9.5e¹³, at least 1e¹⁴, at least 1.5e¹⁴, at least 2e¹⁴, at least 2.5e¹⁴, at least 3e¹⁴, at least 3.5e¹⁴, at least 4e¹⁴, at least 4.5e¹⁴, at least 5e¹⁴, at least 5.5e¹⁴, at least 6e¹⁴, at least 6.5e¹⁴, at least 7e¹⁴, at least 7.5e¹⁴, at least 8e¹⁴, at least 8.5e¹⁴, at least 9e¹⁴, at least 9.5e¹⁴, at least 1e¹⁵, at least 1.5e¹⁵, at least 2e¹⁵, at least 2.5e¹⁵, at least 3e¹⁵, at least 3.5e¹⁵, at least 4e¹⁵, at least 4.5e¹⁵, at least 5e¹⁵, at least 5.5e¹⁵, at least 6e¹⁵, at least 6.5e¹⁵, at least 7e¹⁵, at least 7.5e¹⁵, at least 8e¹⁵, at least 8.5e¹⁵, at least 9e¹⁵, at least 9.5e¹⁵ or, at least 1e¹⁶.

In one embodiment, a high dose of a viral vector is at least 3.4e¹⁴ vg/kg.

The viral vector dose disclosed herein is higher than the highest achievable dose in presence of prednisone that is used to mitigate the toxicity or, to improve the viral transduction efficiency. The viral vector dose is the dose wherein IFNy response to viral capsid is negligible.

In a further embodiment, administration of a viral vector and/or antibiotic as disclosed herein to a subject results in production of a therapeutic protein with a circulatory half-life of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a viral vector and/or antibiotic as disclosed herein to a subject is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Antibiotics of the invention may be administered through any route encompassed by systemic or local administration as defined. For oral administration the antibiotics, e.g., tetracycline members, utilized in this invention may be formulated in the form of tablets, capsules, elixirs or the like. For parenteral administration they may be formulated into solutions or suspensions or intramuscular injections, or additionally, the tetracyclines of the present invention may be reasonably incorporated into a polymer carrier delivery system for use topically or locally. The dosage of tetracyclines administered in the present invention is also additionally dependent upon the age and weight of the animal species being treated, the mode of administration, and the type and severity of the excess collagenase induced disease being treated.

For instance, the normal and usual dose of tetracycline is, orally, the equivalent of 250 mg. It is specifically contemplated herein that tetracycline be administered to a pediatric subject (<18 years of age) at 25 mg/kg-50 mg/kg daily, divided into 2-4 doses per day, and to an adult subject (>18 years of age) at 1-2 gm daily, divided into 2-4 doses per day. For rolitetracycline, the usual dose range is intramuscularly, 150-350 mg every 12 hours, and intravenously, 350-700 mg every 12 hours. For chlortetracycline, the usual daily dose range is 250-500 mg. When oxytetracycline is utilized the usual dose is, orally, 250 mg 4 times daily; intra-muscularly, 100 mg 2 or 3 times daily; and intravenously, 250-500 mg over a period of ½ to 1 hour twice daily. The usual dose range is orally, 1 to 4 g daily; intramuscularly, 200-500 mg daily; intravenously 500 mg to 2 g daily. For methacycline the usual dose is 600 mg of the hydrochloride salt or 560 mg of methacycline base, daily in divided doses. When demeclocycline is utilized, the usual dose is 600 mg daily in 4 divided doses of 150 mg or 2 divided doses of 300 mg each, and the usual dose range is from 150-900 mg per day. Doxycycline is typically utilized in a dosage of 100 mg every 12 hours during the first day of treatment followed by a maintenance dose of 100 mg daily.

Further, Azithromycin is typically utilized in a dosage of 500 mg for the first day of treatment followed by a maintenance dose of 250 mg daily for four days. It is specifically contemplated herein that azithromycin be administered to a pediatric subject (<18 years of age) at 10 mg/kg on the first day of treatment, followed by daily maintenance doses of 5 mg/kg, and to an adult subject (>18 years of age) at 500 mg on the first day of treatment, followed by daily maintenance doses of 250 mg. For clarithromycin, the usual dosage range is orally 250-500 mg every 12 hours or 1000 mg once daily for 7-14 days. It is specifically contemplated herein that clarithromycin be administered to a pediatric subject (<18 years of age) at 7.5 mg/kg twice daily, and to an adult subject (>18 years of age) at 250 mg-500 mg twice daily. For erythromycin, the usual dosage range is orally 500 mg to Ig every 12 h or 250 mg to 1 gm every 6 hours; or intravenously 250 mg to 1 gm every 6 hours. The maximum dose of erythromycin is 4 grams daily. For telithromycin, the usual dosage range is orally 800 mg, taken once every 24 hours, for 7-10 days.

In one embodiment, the dosage of a given antibiotic is at least 10 mg/day, at least 20 mg/day, at least 30 mg/day, at least 40 mg/day, at least 50 mg/day, at least 60 mg/day, at least 80 mg/day, at least 100 mg/day, at least 200 mg/day, at least 300 mg/day, at least 400 mg/day, at least 500 mg/day, at least 600 mg/day, at least 700 mg/day, at least 800 mg/day, at least 900 mg/day, at least 1 gram/day, at least 1.1 gram/day, at least 1.2 gram/day, at least 1.3 gram/day, at least 1.4 gram/day, at least 1.5 gram/day, at least 1.6 gram/day, at least 1.7 gram/day, at least 1.8 gram/day, at least 1.9 gram/day, at least 2 gram/day, at least 2.1 gram/day, at least 2.2 gram/day, at least 2.3 gram/day, at least 2.4 gram/day, at least 2.5 gram/day, at least 2.6 gram/day, at least 2.7 gram/day, at least 2.8 gram/day, at least 2.9 gram/day or more.

Steroids of the invention may be administered through any route encompassed by systemic or local administration as defined. For example, steroids of the invention may be applied locally to the skin, applied locally to the eye, ingested orally, inhaled directly into the lungs, injected into a vein or muscle, or injected directly into inflamed joints. Steroids that may be administered by an oral route include, but are not limited to the following steroids: betamethasone, budesonide, cortisone, dexamethasone, hydrocortisone, methylprednisolone, prednisolone, prednisone, triamcinolone, a combination of two or more of these steroids, and commercial products of these steroids. Steroids that may be administered by a parenteral route, such as parenteral injection, include, but are not limited to the following steroids: betamethasone, cortisone, dexamethasone, hydrocortisone, methylprednisolone, prednisolone, triamcinolone, a combination of two or more of these steroids, and commercial products of these steroids. Steroids that may be administered by inhalation include, but are not limited to the following steroids: beclomethasone, budesonide, flunisolide, fluticasone, mometasone, triamcinolone, a combination of two or more of these steroids, and commercial products of these steroids. Steroids that may be administered by a topical route include, but are not limited to the following steroids: alclometasone, amcinonide, augmented betamethasone, betamethasone, clobetasol, clocortolone, desonide, desoximetasone, dexamethasone, diflorasone, flucinolone, fluocinonide, flurandrenolide, fluticasone, halcinonide, halobetasol, hydrocortisone, methylprednisolone, mometasone, prednicarbate, triamcinolone, a combination of two or more of these steroids, and commercial products of these steroids. One of skill in the art would understand that a particular steroid may be applied by more than one route, e.g. a steroid utilized in a topical formulation may be adapted for intravenous or oral administration.

One of ordinary skill in the art would understand that steroids have various medical uses, including but not limited to: (1) anti-inflammatory uses, e.g. betamethasone, budesonide, cortisone, dexamethasone, hydrocortisone, methylprednisolone, prednisolone, prednisone, and triamcinolone; (2) antiemetic uses, e.g. dexamethasone, hydrocortisone, and prednisone; (3) diagnostic uses, e.g. dexamethasone, as used to detect Cushing's syndrome; and (4) immunosuppressant uses, e.g. betamethasone, cortisone, dexamethasone, hydrocortisone, methylprednicolone, prednisolone, prednisone, and triamcinolone. Moreover, one of ordinary skill in the art would understand that corticosteroid drugs can be used as ingredients contained in eye products (to treat various eye conditions), inhalers (to treat asthma or bronchial disease), nasal drops and sprays (to treat various nasal conditions), and topical products such as ointments and creams (to treat various skin conditions).

One of ordinary skill in the art would understand that potencies may vary among steroids. For example, as associated with systemic administration, betamethasone and dexamethasone exhibit high overall potencies and high anti-inflammatory potencies; methylprednisolone, triamcinolone, prednisolone, and prednisone exhibit medium overall potencies and medium anti-inflammatory potencies; and hydrocortisone and cortisone exhibit low overall potencies and anti-inflammatory potencies.

In one embodiment, a low dose steroid is administered at least 40 mg/day. In one embodiment, a low dose steroid is administered at least 60 mg/day.

In one embodiment, the steroid is administered to the subject once. In one embodiment, the steroid is administered to the subject at least twice. For example, a subject can be administered the steroid at least 1, 2, 3, 4, 5, times per hour, at least 1, 2, 3, 4, 5, 7, 8, 9, 10, or more times per day, at least 1, 2, 3, 4, 5, 7, 8, 9, 10, or more times per week, or at least 2, 3, 4, 5, 7, 8, 9, 10, or more times per month. In one embodiment, the steroid is administered at least once daily for at least 1, 2, 3, 4, 5, 6, 7, 8, 9, 10, 11, 12, 13, 14, 15 or more weeks. In one embodiment, the steroid no longer administered to the subject when adverse effects of the steroid present in the subject. In one embodiment, when the steroid is administered more than once, the dosage of the steroid is the same for each administration, e.g., the steroid is administered at 60 mg/day for each administration. In one embodiment, when the steroid is administered more than once, the dosage of the steroid is different for at least two administrations, e.g., the steroid is administered at 60 mg/day for the first administration, and at 55 mg/day for each subsequent administration.

It is preferred that stopping administration of a steroid is done via tapering, e.g., in a step-wise decrease in dosage over time. In one embodiment, the steroid, e.g., prednisone, is administered for 15 weeks following administration of a viral vector, such that the steroid is administered at 60 mg daily for weeks 1-4 post administration of the viral vector, at 55 mg daily during week 5 post administration, at 50 mg during week 6 post-administration, at 45 mg during week 7 post-administration, at 40 mg during week 8 post-administration, at 35 mg during week 9 post-administration, at 30 mg during week 10 post-administration, at 25 mg during week 11 post-administration, at 20 mg during week 12 post-administration, at 15 mg during week 13 post-administration, at 10 mg during week 14 post-administration, and at 5 mg during week 15 post-administration.

One of ordinary skill in the art would understand that the duration of biological effects elicited by administered steroids may vary among different steroids associated with their respective half-lives. For example, betamethasone and dexamethasone exhibit long half-lives; methylprednisolone, prednisolone, and prednisone exhibit medium half-lives; and cortisone and hydrocortisone exhibit short half-lives. One of skill would understand that the duration of biological effects associated with the half-life of an individual steroid includes the duration of anti-inflammatory effects.

### Delivery of Viral Vector to Target Cells

A viral vector may be delivered to target cells by various available means in the art. Methods of delivery of nucleic acids include, without limitation infection by particles, lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Delivery can be to cells (e.g., in vitro or ex vivo administration) or target tissues (e.g., in vivo administration).

In one embodiment, the viral vector is administered to a cell by transfection. Transfection methods useful for the methods described herein include, but are not limited to, lipid-mediated transfection, cationic polymer-mediated transfection, or calcium phosphate precipitation. Transfection agents suitable for use with the invention include transfection agents that facilitate the introduction of RNA, DNA and proteins into cells. Exemplary transfection reagents include TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.).

In another embodiment, the viral vector is administered to a cell by electroporation (e.g., nucleofection). In some embodiments, the nucleic acids described herein are administered to a cell via microfluidics methods known to those of skill in the art.

Liposome-Mediated Delivery

In embodiments, the viral vector is added to liposomes for delivery to a cell. Liposomes are vesicles that possess at least one lipid bilayer. Liposomes are typically used as carriers for drug/therapeutic delivery in the context of pharmaceutical development. Liposomes work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine). cholesteryl sulphate (CS), dipalmitoylphosphatidylglycerol (DPPG), DOPC (dioleoly-sn glycero-phosphatidylcholine) or any combination thereof.

In some aspects, the disclosure provides for a liposome formulation including phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation including a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation including a lipid containing a phosphatidylcholine functional group, cholesterol and a PEG-ylated lipid. In some aspects, the disclosure provides for a liposome formulation including a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g. cholesterol. In some aspects, the liposome formulation comprises DOPC/DEPC; and DOPE.

In some aspects, the disclosure provides for a liposome formulation further comprising one or more pharmaceutical excipients, e.g., sucrose and/or glycine.

In some aspects, the disclosure provides for a liposome formulation that is either unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with the DNA construct obtained by the process of Example 1 or otherwise disclosed herein, by adding a weak base to a mixture having the isolated DNA construct outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g., polyphosphate or sucrose octasulfate.

In other aspects, the disclosure provides for a liposome formulation comprising phospholipids, lecithin, phosphatidylcholine and phosphatidylethanolamine.

In some aspects, the disclosure provides for lipid formulations, for example, lipid nanoparticle formulations, that are useful in delivering the DNA construct. For example, the lipid nanoparticle formulations described in WO2017/173054, the contents of which are incorporated herein by reference in its entirety, are contemplated for use with the methods and compositions described herein.

Target Cells

The agents to the present invention provide a means for delivering nucleic acids into a broad range of cells, including dividing and non-dividing cells. In one embodiment, the cells are genetically deficient. In one embodiment, the cells are diseased.

The cell(s) into which the viral vector construct is introduced can be of any type, including but not limited to neural cells (including cells of the peripheral and central, nervous systems, in particular, brain, cells such as neurons and oligodendrocytes), lung cells, cells of the eye (including retinal cells, retinal pigment epithelium, and corneal cells), blood vessel cells (e.g., endothelial cells, intimal cells), epithelial cells (e.g., gut and respiratory epithelial cells), muscle, cells (e.g., skeletal muscle cells, cardiac muscle cells, smooth muscle cells and/or diaphragm muscle cells), dendritic cells, pancreatic cells, (including islet cells), hepatic cells, kidney cells, myocardial cells, bone cells (e.g., bone marrow stem cells), hematopoietic stem cells, spleen cells, keratinocytes, fibroblasts, endothelial cells, prostate cells, germ cells, and the, like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin.

### Diseases

Gene transfer has substantial potential use for understanding and providing therapy for disease states. There are a number of inherited diseases in which defective genes are known and have been cloned. In general, the disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, viral vectors according to the present invention permit the treatment and/or prevention of genetic diseases. Exemplary protocols for administering a viral vector for treatment of a disease are described in, e.g., NIH RAC Protocol Number (GemCris): 1320-1275; and Gernoux, G., et al. Human Gene Therapy (2017), the contents of which are incorporated herein by reference in their entireties.

The viral vector according to the present invention may also be employed to provide a functional RNA to a cell in vitro or in vivo. Expression of the functional RNA in the cell, for example, can diminish expression of a particular target protein by the cell. Accordingly, functional RNA can be administered to decrease expression of a particular protein in a subject in need thereof. Functional RNA can also be administered to cells in vitro to regulate gene expression and/or cell physiology, e.g., to optimize cell or tissue culture systems or in screening methods. In certain embodiments, the therapeutic targets a protein for correction of a dysregulated cellular pathway of a disease state.

In general, the viral of the present invention can be employed to deliver a nucleic acid, e.g., a therapeutic gene, encoding a polypeptide or functional RNA to treat and/or prevent any disease state for which it is beneficial to deliver a therapeutic polypeptide or functional RNA. Illustrative disease states include, but are not limited to those listed in Table 2.

TABLE 2
Exemplary diseases and disorders.

Amyotrophic Lateral Sclerosis; endotoxemia; atherosclerotic vascular disease is coronary
artery disease; stent restenosis; carotid metabolic disease; stroke; acute myocardial infarction;
heart failure; peripheral arterial disease; limb ischemia; vein graft failure; AV fistula failure;
Crohn's disease; ulcerative colitis; ileitis and enteritis; vaginitis; psoriasis and inflammatory
dermatoses such as dermatitis; eczema; atopic dermatitis; allergic contact dermatitis; urticaria;
vasculitis; spondyloarthropathies; scleroderma; respiratory allergic diseases such as asthma;
allergic rhinitis; hypersensitivity lung diseases; arthritis (e.g. rheumatoid and psoriatic);
eczema; psoriasis; osteoarthritis; multiple sclerosis; systemic lupus erythematosus; diabetes
mellitus; glomerulonephritis; graft rejection (including allograft rejection and graft-v-host
disease) or rejection of an engineered tissue; infectious diseases; myositis; inflammatory CNS
disorders; stroke; closed-head injuries; neurodegenerative diseases; Alzheimer's disease;
encephalitis; meningitis; osteoporosis; gout; hepatitis; hepatic veno-occlusive disease (VOD);
hemorrhagic cystitis; nephritis; sepsis; sarcoidosis; conjunctivitis; otitis; chronic obstructive
pulmonary disease; sinusitis; Bechet's syndrome; graft-versus-tumor effect; mucositis;
appendicitis; ruptured appendix; peritonitis; aortic valve disease; mitral valve disease; Rett's
syndrome; tuberous sclerosis; phenylketonuria; Smith-Lemli-Opitz syndrome and fragile X
syndrome; Parkinson's disease; Aicardi-Goutières Syndrome; Alexander Disease; Allan-
Hemdon-Dudley Syndrome; POLG-Related Disorders; Alpha-Mannosidosis (Type II and III);
Alström Syndrome; Angelman Syndrome; Ataxia-Telangiectasia; Neuronal Ceroid-
Lipofuscinoses; Beta-Thalassemia; Bilateral Optic Atrophy and (Infantile) Optic Atrophy Type
1; Retinoblastoma (bilateral); Canavan Disease; Cerebrooculofacioskeletal Syndrome 1
[COFS1]; Cerebrotendinous Xanthomatosis; Cornelia de Lange Syndrome; MAPT-Related
Disorders; Genetic Prion Diseases; Dravet Syndrome; Early-Onset Familial Alzheimer Disease;
Friedreich Ataxia [FRDA]; Fryns Syndrome; Fucosidosis; Fukuyama Congenital Muscular
Dystrophy; Galactosialidosis; Gaucher Disease; Organic Acidemias; Hemophagocytic
Lymphohistiocytosis; Hutchinson-Gilford Progeria Syndrome; Mucolipidosis II; Infantile Free
Sialic Acid Storage Disease; PLA2G6-Associated Neurodegeneration; Jervell and Lange-
Nielsen Syndrome; Junctional Epidermolysis Bullosa; Huntington Disease; Krabbe Disease
(Infantile); Mitochondrial DNA-Associated Leigh Syndrome and NARP; Lesch-Nyhan
Syndrome; LIS1-Associated Lissencephaly; Lowe Syndrome; Maple Syrup Urine Disease;
MECP2 Duplication Syndrome; ATP7A-Related Copper Transport Disorders; LAMA2-
Related Muscular Dystrophy; Arylsulfatase A Deficiency; Mucopolysaccharidosis Types I; II
or III; Peroxisome Biogenesis Disorders; Zellweger Syndrome Spectrum; Neurodegeneration
with Brain Iron Accumulation Disorders; Acid Sphingomyelinase Deficiency; Niemann-Pick
Disease Type C; Glycine Encephalopathy; ARX-Related Disorders; Urea Cycle Disorders;
COL1A½-Related Osteogenesis Imperfecta; Mitochondrial DNA Deletion Syndromes; PLP1-
Related Disorders; Perry Syndrome; Phelan-McDermid Syndrome; Glycogen Storage Disease
Type II (Pompe Disease) (Infantile); MAPT-Related Disorders; MECP2-Related Disorders;
Rhizomelic Chondrodysplasia Punctata Type 1; Roberts Syndrome; Sandhoff Disease;
Schindler Disease-Type 1; Adenosine Deaminase Deficiency; Smith-Lemli-Opitz Syndrome;
Spinal Muscular Atrophy; Infantile-Onset Spinocerebellar Ataxia; Hexosaminidase A
Deficiency; Thanatophoric Dysplasia Type 1; Collagen Type VI-Related Disorders; Usher
Syndrome Type I; Congenital Muscular Dystrophy; Wolf-Hirschhom Syndrome; Lysosomal
Acid Lipase Deficiency; and Xeroderma Pigmentosum.

The therapeutic gene described herein modulates, e.g., increases or decreases, the expression of a disease gene. In one embodiment, the therapeutic gene alters (e.g., increases or decreases) the expression of a disease gene or gene product therefrom. For example, expression of the therapeutic gene in a cell increases the expression of a disease gene by at least 5%, 10%, 15%, 20%, 25%, 30%, 35%, 40%, 45%, 50%, 55%, 60%, 65%, 70%, 75%, 80%, 85%, 90%, 95%, 99%, or more as compared to a reference level, or at least 2-fold, 5-fold, 10-fold, 20-fold, 30-fold, 40-fold, 50-fold, 60-fold, 70-fold, 80-fold, 90-fold, 100-fold, 150-fold, 200-fold, 250-fold, 300-fold, 350-fold, 400-fold, 450-fold, 500-fold, or more as compared to a reference level. Alternatively, expression of the therapeutic gene in a cell decreases the expression of a disease gene by at least 500, 10%, 15%, 20%, 25%, 30%, 35%, 40%, 45%, 50%, 55%, 60%, 65%, 70%, 75%, 80%, 85%, 90%, 95%, 99% or more as compared to a reference level. As used herein, “reference level” refers to the expression level of the disease gene in an otherwise identical sample that is not co-administered the viral vector and antibiotic, or is administered the viral vector without co-administration of the antibiotic. One skilled in the art can assess the level of a disease gene or gene product therefrom using standard techniques, for example, using PCR-based assays or western blotting to measure mRNA or protein levels, respectively. Illustrative disease genes include, but are not limited to those listed in Table 1.

TABLE 1
Exemplary disease genes.

1p36; 18p; 6p21.3; 14q32; AAAS; FGD1; EDNRB; CP (3p26.3); LMBR1; COL2A1
(12q13.11); 4p16.3; HMBS; ADSL; ABCD1; JAG1; NOTCH2; TP63; TREX1; RNASEH2A;
RNASEH2B; GAA; RNASEH2C; SAMHD1; ADAR; IFIH1; GFAP; HGD; 10q26.13;
ATP1A3; ALMS1; ALAD; FGFR2; VPS33B; ATM; PITX2; FOXO1A; FOXC1; PAX6;
10q26; FGFR2; IGF-2; CDKN1C; H19; KCNQ1OT1; BTD; BCS1L; 15q26.1; 17 FLCN;
ATP2A1; MAOA; NOTCH3; HTRA1; X 17q24.3-q25.1; ASPA; RAB23; SNAP29; FTR
(7q31.2); PMP22; MFN2; CHD7; LYST; RUNX2; ERCC6; ERCC8; X RPS6KA3; COH1;
COL11A1; COL11A2; COL2A1; NTRK1; PTEN; CPOX; 14q13-q21; 5p; 16q12; FGFR2;
FGFR3; FGFR3; ATP2A2; Xp11.22 CLCN5; OCRL; WT1; 18q; 22q11.2; HSPB8; HSPB1;
HSPB3; GARS; REEP1; IGHMBP2; SLC5A7; DCTN1; TRPV4; SIGMAR1; COL1A1;
COL1A2; COL3A1; COL5A1; COL5A2; TNXB; ADAMTS2; PLOD1; B4GALT7; DSE;
EMD; LMNA; SYNE1; SYNE2; FHL1; TMEM43; FECH; FANCA; FANCB; FANCC;
FANCD1; FANCD2; FANCE; FANCF; FANCG; FANCI; FANCJ; FANCL; FANCM;
FANCN; FANCP; FANCS; RAD51C; XPF; GLA (Xq22.1); APC; IKBKAP; MYCN; MED12;
FXN; GALT; GALK1; GALE; GBA (1); PAX6; GCDH; ETFA; ETFB; ETFDH; BCS1L;
MYO5A; RAB27A; MLPH; ATP2C1 (3); ABCA12; HFE; HAMP; HFE2B; TFR2; TF; CP;
FVIII; UROD; 3q12; ENG; ACVRL1; MADH4; GNE; MYHC2A; VCP; HNRPA2B1;
HNRNPA1; EXT1; EXT2; EXT3; HPS1; HPS3; HPS4; HPS5; HPS6; HPS7; AP3B1; PMP22;
NODAL; NKX2-5; ZIC3; CCDC11; CFC1; SESN1; CBS (gene); HD; IDS; IDUA; AASS;
AGXT; GRHPR; DHDPSL; ABCA1; COL2A1; FGFR3 (4p16.3); 20q11.2; IKBKG (Xq28);
TBX4; 15q11-14; FGFR2; INPP5E; TMEM216; AHI1; NPHP1; CEP290; TMEM67;
RPGRIP1L; ARL13B; CC2D2A; OFD1; TMEM138; TCTN3; ZNF423; AMRC9; ALS2;
COL2A1; PDGFRB; GAL; ATP13A2; LCAT; HPRT (X); TP53; MSH2; MLH1; MSH6;
PMS2; PMS1; TGFBR2; MLH3; RYR1 (19q13.2); BCKDHA; BCKDHB; DBT; DLD; ARSB;
20 q13.2-13.3; XK (X); AP1S1; MEFV; ATP7A (Xq21.1); MMAA; MMAB; MMACHC;
MMADHC; LMBRD1; MUT; RAB3GAP (2q21.3); ASPM (1q31); GALNS; GLB1; ZEB2 (2);
FGFR3; MEN1; RET; MSTN; DMPK; CNBP; HYAL1; 17q11.2; SMPD1; NPA; NPB; NPC1;
NPC2; GLDC; AMT; GCSH; PTPN11; KRAS; SOS1; RAF1; NRAS; HRAS; BRAF; SHOC2;
MAP2K1; MAP2K2; CBL; RELN; RAG1; RAG2; COL1A1; COL1A2; IFITM5; PANK2
(20p13-p12.3); UROD; PDS; STK11; FGFR1; FGFR2; PAH; AASDHPPT; TCF4 (18); PKD1
(16) or PKD2 (4); DNAI1; DNAH5; TXNDC3; DNAH11; DNAI2; KTU; RSPH4A; RSPH9;
LRRC50; PROC; PROS1; ABCC6; RP1; RP2; RPGR; PRPH2; IMPDH1; PRPF31; CRB1;
PRPF8; TULP1; CA4; HPRPF3; ABCA4; EYS; CERKL; FSCN2; TOPORS; SNRNP200;
PRCD; NR2E3; MERTK; USH2A; PROM1; KLHL7; CNGB1; TTC8; ARL6; DHDDS;
BEST1; LRAT; SPARA7; CRX; MECP2; ESCO2; CREBBP; HEXB; SGSH; NAGLU;
HGSNAT; GNS; HSPG2; COL2A1; FBN1; 11p15; Xp11.22; PHF8; ABCB7; SLC25A38;
GLRX5; GUSB; DHCR7; 17p11.2; ATXN1; ATXN2; ATXN3; PLEKHG4; SPTBN2;
CACNA1A; ATXN7; ATXN8OS; ATXN10; TTBK2; PPP2R2B; KCNC3; PRKCG; ITPR1;
TBP; KCND3; FGF14; FGFR3; ABCA4; CNGB3; ELOVL4; PROM1; COL11A1; COL11A2;
COL2A1; COL9A1; COL2A1; HEXA (15); GCH1; PCBD1; PTS; QDPR; MTHFR; DHFR;
FGFR3; 5q32-q33.1 (TCOF1; POLR1C; or POLR1D); TSC1; TSC2; MYO7A; USH1C;
CDH23; PCDH15; USH1G; USH2A; GPR98; DFNB31; CLRN1; PPOX; VHL; PAX3; MITF;
WS2B; WS2C; SNAI2; EDNRB; EDN3; SOX10; COL11A2; ATP7B; C2ORF37 (2q22.3-
q35); 4p16.3; 15 ERCC4; CENPVL1; CENPVL2; GSPT2; MAGED1; ALAS2 (X); PEX1;
PEX2; PEX3; PEX5; PEX6; PEX10; PEX12; PEX13; PEX14; PEX16; PEX19; and PEX26

In one embodiment, the heterologous nucleic acid further encode reporter polypeptides (e.g., an enzyme). Reporter polypeptides are known in the art and include, but are not limited to, Green Fluorescent Protein, β-galactosidase, alkaline phosphatase, luciferase, and chloramphenicol acetyltransferase gene.

In one embodiment, the heterologous nucleic acid is operatively linked to a control element, such as transcription/translation control signals, origins of replication, polyadenylation signals, internal ribosome entry sites (IRES), promoters, and/or enhancers, and the like.

Alternatively, in particular embodiments of this invention, the heterologous nucleic acid may encode an antisense nucleic acid, a ribozyme (e.g., as described in U.S. Pat. No. 5,877,022), RNAs that effect spliceosome-mediated trans-splicing (see, Puttaraju et al. (1999) Nature Biotech. 17:246; U.S. Pat. Nos. 6,013,487; 6,083,702), interfering RNAs (RNAi) including siRNA, shRNA or miRNA that mediate gene silencing (see, Sharp et al. (2000) Science 287:2431), and other non-translated RNAs, such as “guide” RNAs (Gorman et al. (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see e.g., Andino et al. J. Gene Med. 10:132-142 (2008) and Li et al. Acta Pharmacol Sin. 26:51-55 (2005)); phospholamban inhibitory or dominant-negative molecules such as phospholamban S16E (e.g., to treat cardiovascular disease, see e.g., Hoshijima et al. Nat. Med. 8:864-871 (2002)), RNAi to adenosine kinase (e.g., for epilepsy), and RNAi directed against pathogenic organisms and viruses (e.g., hepatitis B and/or C virus, human immunodeficiency virus, CMV, herpes simplex virus, human papilloma virus, etc.).

In one embodiment, the viral vector expresses an immunogenic polypeptide, e.g., for vaccination. An immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env gene products). The immunogenic polypeptide can also be an arenavirus immunogen (e.g., Lassa fever virus immunogen, such as the Lassa fever virus nucleocapsid protein and/or the Lassa fever envelope glycoprotein), a poxvirus immunogen (e.g., a vaccinia virus immunogen, such as the vaccinia L1 or L8 gene product), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpesvirus immunogen (e.g., CMV, EBV, HSV immunogens) a mumps virus immunogen, a measles virus immunogen, a rubella virus immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell. Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al. (1994) Proc. Natl. Acad. Sci. USA 91:3515; Kawakami et al. (1994) J. Exp. Med., 180:347; Kawakami et al. (1994) Cancer Res. 54:3124), MART-1, gp100 MAGE-1, MAGE-2, MAGE-3, CEA, TRP-1, TRP-2, P-15, tyrosinase (Brichard et al. (1993) J. Exp. Med. 178:489); HER-2/neu gene product (U.S. Pat. No. 4,968,603), CA125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (PCT Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

The viral vectors of the present invention can be employed to deliver a heterologous nucleic acid encoding a polypeptide or functional RNA to treat and/or prevent any disease state for which it is beneficial to deliver a therapeutic polypeptide or functional RNA.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.”

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The invention is further illustrated by the following examples, which should not be construed as further limiting.

Various embodiments are the invention described herein can further be described in the following set of numbered paragraphs.
1. A method of reducing toxicity in treating a subject with recombinant viral vector, the method comprising co-administration of an antibiotic and viral vector to the subject.
2. The method of paragraph 1, wherein the co-administration of the antibiotic is
   a. prior to administration of the viral vector;
   b. at substantially the same time as administration of the viral vector; and/or
   c. after administration of the viral vector.
3. The method of any of the preceding paragraphs, wherein the subject is further administered prednisone 4. The method of any of the preceding paragraphs, wherein the subject is not administered prednisone.
5. The method of any of the preceding paragraphs, wherein the antibiotic is a member of the tetracycline family of antibiotics, or a member of the macrolides family of antibiotics.
6. The method of any of the preceding paragraphs, wherein the tetracycline member is selected from the group consisting of Tetracycline, Chlortetracycline, Oxytetracycline, Demeclocycline, Lymecycline, Meclocycline, Methacycline, Minocycline, Rolitetracycline, Doxycycline, Tigecycline, Eravacycline, Sarecycline, and Omadacycline.
7. The method of any of the preceding paragraphs, wherein the macrolide member is selected from the group consisting of Clarithromycin, Azithromycin, Fidoximycin, and Erythromycin.
8. The method of any of the preceding paragraphs, wherein the viral vector is selected from the list consisting of an adeno-associated viral (AAV) vector, an adenovirus vector, a lentivirus vector, a retrovirus vector, a herpesvirus vector, an alphavirus vector, a poxvirus vector, a baculovirus vector, and a chimeric virus vector.
9. The method of any of the preceding paragraphs, wherein the viral vector is administered at a dose of greater than 1.5e¹².
10. The method of any of the preceding paragraphs, wherein the viral vector genome comprises
   a. 5′ and 3′ AAV inverted terminal repeats (ITR) sequences, and
   b. located between the 5′ and 3′ ITRs, a heterologous nucleic acid sequence encoding a therapeutic gene, wherein the heterologous nucleic acid is operatively linked to a promoter.
11. The method of any of the preceding paragraphs, wherein the viral vector comprises a capsid protein selected from the group consisting of hybrid, chimeric, mosaic, polyploid and haploid group of rAAVs.
12. The method of any of the preceding paragraphs, wherein the ITR is a wild-type (WT) ITR, a mutant ITR, or a synthetic ITR.
13. The method of any of the preceding paragraphs, wherein the therapeutic gene alters expression of a disease gene.
14. The method of any of the preceding paragraphs, wherein the therapeutic gene increases or decreases expression of a disease gene.
15. The method of any of the preceding paragraphs, wherein the disease gene is selected from the group consisting of those listed in Table 1.
16. The method of any of the preceding paragraphs, wherein the subject is at risk of having, or has been diagnosed as having a disease selected from the group consisting of those listed in Table 2.
17. A method enabling administration of recombinant viral vector to a subject in the absence of prednisone, or in the presence of a low dose of prednisone, the method comprising co-administration of an antibiotic and at least 1.5e¹² recombinant viral vector.
18. The method of any of the preceding paragraphs, wherein the co-administration of the antibiotic is
   a. prior to administration of the viral vector;
   b. at substantially the same time as administration of the viral vector; and/or
   c. after administration of the viral vector.
19. The method of any of the preceding paragraphs, wherein the antibiotic is a member of the tetracycline family of antibiotics, or a member of the macrolides family of antibiotics.
20. The method of any of the preceding paragraphs, wherein the tetracycline member is selected from the group consisting of Tetracycline, Chlortetracycline, Oxytetracycline, Demeclocycline, Lymecycline, Meclocycline, Methacycline, Minocycline, Rolitetracycline, Doxycycline, Tigecycline, Eravacycline, Sarecycline, and Omadacycline.
21. The method of any of the preceding paragraphs, wherein the macrolide member is selected from the group consisting of Clarithromycin, Azithromycin, Fidoximycin, and Erythromycin.
22. The method of any of the preceding paragraphs, wherein the tetracycline family of antibiotic is administered in combination with another antibiotic selected from either tetracycline family or the macrolides family of antibiotics.
23. The method of any of the preceding paragraphs, wherein the antibiotic is administered at least once.
24. The method of any of the preceding paragraphs, wherein the antibiotic is administered at least twice.
25. The method of any of the preceding paragraphs, wherein the viral vector is selected from the list consisting of an AAV vector, an adenovirus vector, a lentivirus vector, a retrovirus vector, a herpesvirus vector, an alphavirus vector, a poxvirus vector, a baculovirus vector, and a chimeric virus vector.
26. The method of any of the preceding paragraphs, wherein administration of the antibiotic and viral vector is systemic.
27. The method of any of the preceding paragraphs, wherein administration of the antibiotic is systemic and administration of the viral vector is local.
28. A composition comprising a viral vector and an antibiotic.
29. The composition of any of the preceding paragraphs, wherein the composition further comprises prednisone 30. The composition of any of the preceding paragraphs, wherein the composition does not comprise prednisone.
31. A pharmaceutical composition comprising a viral vector and an antibiotic.
32. The composition of any of the preceding paragraphs, wherein the composition further comprises prednisone.
33. The composition of any of the preceding paragraphs, wherein the composition does not comprise prednisone.
34. The composition of any of the preceding paragraphs, wherein the antibiotic is a member of the tetracycline family of antibiotics, or a member of the macrolides family of antibiotics.
35. The composition of any of the preceding paragraphs, wherein the tetracycline member is selected from the group consisting of Tetracycline, Chlortetracycline, Oxytetracycline, Demeclocycline, Lymecycline, Meclocycline, Methacycline, Minocycline, Rolitetracycline, Doxycycline, Tigecycline, Eravacycline, Sarecycline, and Omadacycline.
36. The composition of any of the preceding paragraphs, wherein the macrolide member is selected from the group consisting of Clarithromycin, Azithromycin, Fidoximycin, and Erythromycin.
37. The composition of any of the preceding paragraphs, wherein the viral vector is administered at a dose of greater than 1.5e¹².
38. The composition of any of the preceding paragraphs, wherein the viral vector is selected from the list consisting of an AAV vector, an adenovirus vector, a lentivirus vector, a retrovirus vector, a herpesvirus vector, an alphavirus vector, a poxvirus vector, a baculovirus vector, and a chimeric virus vector.

### EXAMPLES

### Example 1—Injection of Piglet with (1) an Antibiotic and (2) an Antibiotic and Low-Dose Steroid Prior to Administration of Adeno Associated Virus (AAV) Particles Containing Human Acid Alpha Glucosidase Nucleic Acid Results in Enhanced Expression of Human Acid Alpha Glucosidase

The effect of pretreating piglets with an antibiotic or an antibiotic and low-dose steroid prior to administration of adeno associated virus (AAV) particles operably encoding human glucosidase alpha, acid (GAA) is examined.

The rAAV encodes human GAA (“rAAV-GAA”), which is driven by the cytomegalovirus (CMV) early promoter. The virus preparations are highly purified by two rounds of cesium chloride banding and particle titers are determined as previously described. See, e.g., Nyberg-Hoffman, et al, Nat. Med. 3: 808-811 (1997); Chardonnet and Dales, Virology 40: 462-477 (1970).

Piglets are injected intramuscularly with various doses of rAAV-GAA in 100 μl phosphate buffered saline (“PBS”). Doses and virus constructs are as described below. Blood is aseptically obtained through the jugular vein on day 3 or 4 as specified for rAAV-GAA assays, sera are prepared, and samples are stored at −80° C.

GAA levels are measured by an ELISA assay. Ninety-six-well plates are coated overnight at 4° C. with an anti-human GAA antibody, (ab102815; Abcam, Cambridge, Mass.). The antibody is used at 10 μg/ml in the coating buffer containing 50 mM sodium bicarbonate/carbonate, 0.2 mM MgCl₂, and 0.2 mM CaCl₂ (pH 9.6). After the plates are blocked with 0.5% non-fat dry milk in PBS for 1 hr at room temperature, samples or protein standards (AVONEX™, Biogen Idee), diluted in 10% normal mouse serum/0.5% non-fat dry milk/0.05% Tween-20 in PBS, are then added. After capture for 1.5 hr at room temperature, the plates are washed and successively incubated at room temperature for 1 hr with horseradish peroxidase (“HRP”)-conjugated goat anti-pig antibody (Abcam, 1:5000 dilution). Following a final wash, substrate solution (4.2 mM tetramethylbenzidine, 0.1 M sodium acetate-citric acid, pH 4.9) is then added. The reaction is stopped by the addition of 2 M hydrogen persulfate (“H₂SO₄”) and absorbance is measured at 450 nm.

In one set of experiments, female piglets (n=5/group) are intramuscularly injected with increasing doses of tetracycline or tetracycline and low-dose prednisolone six hours prior to administration of the rAAV-GAA. The concentration of GAA in the sera is determined by ELISA on day 4 post vector dosing.

Increasing doses of tetracycline or tetracycline and low-dose prednisolone enhanced GAA expression in a dose-dependent manner. Pretreatment with increasing doses of tetracycline from 5 mg/kg/day-50 mg/kg/day alone or in combination with low-dose prednisone from 1 mg/kg/day to 25 mg/kg/day is able to increase the expression levels of GAA upon administration of a dose of AAV vector (2×10¹² particles per piglet) in a dose-dependent manner. As compared to animals in the control group pretreated with PBS, there is an observed increase in the levels of serum GAA detected of up to eight-fold when animals are pretreated with tetracycline or tetracycline and low-dose prednisolone combination.

Further, the duration in which GAA is expressed is assessed in animals that are pretreated with tetracycline alone or in combination with low-dose prednisone. A series of sera samples are measured via ELISA at 1, 2, 4, 6, 10, 14, 18, and 24 weeks post vector dosing to determine the concentration of GAA in the sera. Pretreatment with increasing doses of tetracycline from 5 mg/kg/day-50 mg/kg/day alone or in combination with low-dose prednisone from 1 mg/kg/day to 25 mg/kg/day is able to greatly increase the duration in which detectable GAA levels are observed following administration of a single dose of rAAV-GAA (2×10¹² particles per piglet). As compared to animals in the control group pretreated with PBS, detectable GAA levels is observed up to 3-fold longer when animals are pretreated with tetracycline or tetracycline and low-dose prednisolone combination.

These results indicated that pretreatment with an antibiotic alone or in combination with a low-dose steroid can dramatically enhance the resulting levels of GAA expressed from a rAAV vector.

Treatment with piglets with tetracycline alone or in combination with tapering dose of prednisone result in enhanced GAA expression as compared to the control piglets that did not receive the treatment. Piglets are pretreated with 25 mg/kg/day prednisolone 24 hours prior to rAAV-GAA administration and then continued for 4 weeks. Then each week the prednisone is tapered by 5 mg/kg until no prednisone and continued for 15 weeks post AAV administration (2×10¹² particles per piglet). Tetracycline is then administered at 15 mg/kg/day for 16-24 weeks. A series of sera samples are assessed to measure GAA levels by ELISA method. GAA level is significantly enhanced when piglets are treated with tapering dose of prednisone along with tetracycline as compared to control piglets that received only PBS. Moreover, there is a time dependent increase of GAA level in sera from week 1, week 4, week 15 and week 24. Other groups of piglets receiving either tetracycline doses selected from ranges of tetracycline between 5 mg/kg/day and 50 mg/kg/day along with tapering dose of prednisone (starting from 25 mg/kg tapering off until no prednisone) or, receiving only tetracycline doses selected from ranges of tetracycline between 5 mg/kg/day and 50 mg/kg/day show GAA levels are increased up to 10 fold compared to control piglets receiving no tetracycline and/or tetracycline combined with steroid treatment.

### Example 2—Pretreatment with an Antibiotic Resulted in Decreased Toxicity Upon Administration of rAAV Vector

The effect of antibiotic pretreatment on the rAAV particle-induced toxicity e.g., inflammatory response and liver enzyme levels (e.g., toxicity) is examined by measuring levels of endogenous cytokines and liver enzymes AST and ALT in animals injected with rAAV8-Luc particles operably encoding luciferase nucleic acid.

Female piglets (n=5) are injected intravenously with either PBS alone (antibiotic control) or 25 mg/kg/day tetracycline in PBS 4 hours prior to intravenous administration of PBS alone (viral control) or 2×10¹² particles of rAAV-Luc vector. Small amounts of blood are collected aseptically obtained through the jugular vein at 30 minutes, 60 minutes, 90 minutes, 120 minutes, 1 day, 3 days, 10 days, 21 days, 28 days, 42 days, 60 days post-rAAV-Luc treatment, and serum is isolated. The serum is then analyzed for an inflammatory cytokine profile using the BD™ Cytometric Bead Array Mouse Inflammation Kit (BD™ CBA Mouse Inflammation Kit, BD Biosciences), specifically measuring levels of IL-2, IL-6, IL-7, IL-10, MCP-I, IFN_{γ}, IP-10 and MIG. Liver transaminases e.g., AST and ALT are also measured at the above mentioned timepoints.

Pretreatment with tetracycline significantly reduce the inflammatory response resulting from administration of rAAV-Luc as seen by reduced level of the IL2, IL-6, IL-7, IL-10, MCP-1, IFN_{γ}, IP-10 and MIG, as compare to the antibiotic control injected animals. As expected, pretreatment with PBS alone followed by another injection of PBS alone or pretreatment with tetracycline followed by an injection of PBS alone resulted in no inflammatory response. In contrast, pretreatment with PBS followed by injection of rAAV-Luc resulted in a significant increase in IL-2, IL-6, IL-7, IL-10, MCP-I, IFN_{γ}, IP-10 and MIG shortly after injection with rAAV-Luc. In contrast, pretreatment with tetracycline followed by injection of rAAV-Luc is able to significantly inhibit increases in the levels of these cytokines. In addition, liver AST/ALT enzyme ratio is greater than 2 in antibiotic control group where animals receive only PBS and not antibiotic, whereas AST/ALT ratio significantly improve in the tetracycline treated group where with time, the AST/ALT ratio decreases and becomes less than 1. These results indicate that pretreatment with a tetracycline can dramatically reduce the toxicity associated with rAAV vector administration.

Finally, it is determined whether administration of an antibiotic can reduce toxicity associated with rAAV vector administration.

### Example 3—Treatment with an Antibiotic Resulted in Reduced Toxicity Following Administration of an rAAV Vector

The effect of antibiotic treatment on rAAV particle-induced toxicity is examined by measuring levels of endogenous cytokines and liver transaminases e.g., AST and ALT in animals injected with rAAV-Luc particles operably encoding luciferase nucleic acid.

Female piglets (n=5) are pretreated with 25 mg/kg/day prednisolone 24 hours prior to rAAV8-luc (2×10¹² particles of rAAV-Luc particles) administration and then continued for 4 weeks. Then each week the prednisone is tapered by 5 mg/kg until no prednisone and continued for 15 weeks post AAV administration (2×10¹² particles per piglet). Tetracycline is then administered at 15 mg/kg/day for 16-24 weeks. Small amounts of blood are collected aseptically obtained through the jugular vein at 2 hrs, 4 hrs, 1 day, 3 days, 10 days, 21 days, 4 weeks, 8 weeks, 15 weeks, 24 weeks post-rAAV-Luc treatment, post-rAAV-Luc treatment, and serum is isolated. Toxicity is confirmed in the control group that did not receive tetracycline or, combination of tetracycline and prednisone, as shown by the increased AST/ALT ratio e.g., ratio value over 2.0 and/or, the increased inflammatory cytokine profile (using the BD™ Cytometric Bead Array Mouse Inflammation Kit; BD™ CBA Mouse Inflammation Kit, BD Biosciences), specifically measuring levels of IL2, IL-6, IL-7, IL-10, MCP-1, IFN_{γ}, IP-10 and MIG.

Liver transaminase e.g., AST/ALT level is significantly reduced and becomes normal with time (e.g., less than 1) when piglets are treated with tapering dose of prednisone along with tetracycline as compared to control piglets that received only PBS. Additionally, increased serum levels of IL2, IL-6, IL-7, IL-10, MCP-1, IFN_{γ}, IP-10 and MIG in the control group not receiving tetracycline and/or prednisone treatment are significantly reduced when treated with tetracycline along with a tapering dose of prednisone. Moreover, there is a time dependent decrease of AST/ALT level in sera from week 1, week 4, week 15 and week 24. Other groups of piglets receiving either tetracycline doses selected from ranges of tetracycline between 5 mg/kg/day and 50 mg/kg/day along with tapering dose of prednisone (starting from 25 mg/kg tapering off until no prednisone) or, receiving only tetracycline doses selected from ranges of tetracycline between 5 mg/kg/day and 50 mg/kg/day show AST/ALT levels are decreased significantly compared to control piglets receiving no tetracycline and/or tetracycline combined with steroid treatment. Thus, these data indicate that administration of tetracycline significantly reduces toxicity resulting from administration of rAAV-Luc.

1. A method of reducing toxicity in treating a subject with recombinant viral vector, the method comprising co-administration of an antibiotic and viral vector to the subject.

2. The method of claim 1, wherein the co-administration of the antibiotic is
a. prior to administration of the viral vector;
b. at substantially the same time as administration of the viral vector; and/or
c. after administration of the viral vector.

3. The method of claim 1, wherein the subject is further administered prednisone

4. The method of claim 1, wherein the subject is not administered prednisone.

5. The method of claim 1, wherein the antibiotic is a member of the tetracycline family of antibiotics, or a member of the macrolides family of antibiotics.

6. The method of claim 5, wherein the tetracycline member is selected from the group consisting of Tetracycline, Chlortetracycline, Oxytetracycline, Demeclocycline, Lymecycline, Meclocycline, Methacycline, Minocycline, Rolitetracycline, Doxycycline, Tigecycline, Eravacycline, Sarecycline, and Omadacycline.

7. The method of claim 5, wherein the macrolide member is selected from the group consisting of Clarithromycin, Azithromycin, Fidoximycin, and Erythromycin.

8. The method of claim 1, wherein the viral vector is selected from the list consisting of an adeno-associated viral (AAV) vector, an adenovirus vector, a lentivirus vector, a retrovirus vector, a herpesvirus vector, an alphavirus vector, a poxvirus vector, a baculovirus vector, and a chimeric virus vector.

9. The method of claim 1, wherein the viral vector is administered at a dose of greater than 1.5e¹².

10. The method of claim 1 or 8, wherein the viral vector genome comprises
a. 5′ and 3′ AAV inverted terminal repeats (ITR) sequences, and
b. located between the 5′ and 3′ ITRs, a heterologous nucleic acid sequence encoding a therapeutic gene, wherein the heterologous nucleic acid is operatively linked to a promoter.

11. The method of claim 1, 8 or 10, wherein the viral vector comprises a capsid protein selected from the group consisting of hybrid, chimeric, mosaic, polyploid and haploid group of rAAVs.

12. The method of claim 10, wherein the ITR is a wild-type (WT) ITR, a mutant ITR, or a synthetic ITR.

13. The method of claim 10, wherein the therapeutic gene alters expression of a disease gene.

14. The method of claim 10, wherein the therapeutic gene increases or decreases expression of a disease gene.

15. The method of any of claims 13-14, wherein the disease gene is selected from the group consisting of those listed in Table 1.

16. The method of claim 1, wherein the subject is at risk of having, or has been diagnosed as having a disease selected from the group consisting of those listed in Table 2.

17. A method enabling administration of recombinant viral vector to a subject in the absence of prednisone, or in the presence of a low dose of prednisone, the method comprising co-administration of an antibiotic and at least 1.5e¹² recombinant viral vector.

18. The method of claim 17, wherein the co-administration of the antibiotic is
a. prior to administration of the viral vector;
b. at substantially the same time as administration of the viral vector; and/or
c. after administration of the viral vector.

19. The method of claim 17 or 18, wherein the antibiotic is a member of the tetracycline family of antibiotics, or a member of the macrolides family of antibiotics.

20. The method of claim 19, wherein the tetracycline member is selected from the group consisting of Tetracycline, Chlortetracycline, Oxytetracycline, Demeclocycline, Lymecycline, Meclocycline, Methacycline, Minocycline, Rolitetracycline, Doxycycline, Tigecycline, Eravacycline, Sarecycline, and Omadacycline.

21. The method of claim 19, wherein the macrolide member is selected from the group consisting of Clarithromycin, Azithromycin, Fidoximycin, and Erythromycin.

22. The method of claim 1 or 17, wherein the tetracycline family of antibiotic is administered in combination with another antibiotic selected from either tetracycline family or the macrolides family of antibiotics.

23. The method of claim 1 or 17, wherein the antibiotic is administered at least once.

24. The method of claim 1 or 17, wherein the antibiotic is administered at least twice.

25. The method of claim 17, wherein the viral vector is selected from the list consisting of an AAV vector, an adenovirus vector, a lentivirus vector, a retrovirus vector, a herpesvirus vector, an alphavirus vector, a poxvirus vector, a baculovirus vector, and a chimeric virus vector.

26. The method of any of claim 1-25, wherein administration of the antibiotic and viral vector is systemic.

27. The method of any of claim 1-25, wherein administration of the antibiotic is systemic and administration of the viral vector is local.

28. A composition comprising a viral vector and an antibiotic.

29. The composition of claim 28, wherein the composition further comprises prednisone

30. The composition of claim 28, wherein the composition does not comprise prednisone.

31. A pharmaceutical composition comprising a viral vector and an antibiotic.

32. The pharmaceutical composition of claim 31, wherein the composition further comprises prednisone.

33. The pharmaceutical composition of claim 31, wherein the composition does not comprise prednisone.

34. The composition of any of claims 28-33, wherein the antibiotic is a member of the tetracycline family of antibiotics, or a member of the macrolides family of antibiotics.

35. The composition of claim 34, wherein the tetracycline member is selected from the group consisting of Tetracycline, Chlortetracycline, Oxytetracycline, Demeclocycline, Lymecycline, Meclocycline, Methacycline, Minocycline, Rolitetracycline, Doxycycline, Tigecycline, Eravacycline, Sarecycline, and Omadacycline.

36. The composition of claim 34, wherein the macrolide member is selected from the group consisting of Clarithromycin, Azithromycin, Fidoximycin, and Erythromycin.

37. The composition of any of claim 28 or 31, wherein the viral vector is administered at a dose of greater than 1.5e¹².

38. The composition of claim 28 or 31, wherein the viral vector is selected from the list consisting of an AAV vector, an adenovirus vector, a lentivirus vector, a retrovirus vector, a herpesvirus vector, an alphavirus vector, a poxvirus vector, a baculovirus vector, and a chimeric virus vector.


SEQUENCE LISTING (42 sequences)

SEQ ID NO: 1-35 - skipped (35 entries)

SEQ ID NO: 36 - DNA, 167 bp, Artificial Sequence
  Description of Artificial Sequence: Synthetic polynucleotide
aggaacccctagtgatggagttggccactccctctctgcgcgctcgctcgctcactgaggcaatttgataaaaatcgtcaaattataaacaggctttgcctgtttagcctcagtgagcgagcgagcgcgcagagagggagtggccaactccatcactaggggttcct

SEQ ID NO: 37 - DNA, 143 bp, Artificial Sequence
  Description of Artificial Sequence: Synthetic polynucleotide
aggaacccctagtgatggagttggccactccctctctgcgcgctcgctcgctcactgagggataaaaatccaggctttgcctgcctcagtgagcgagcgagcgcgcagagagggagtggccaactccatcactaggggttcct

SEQ ID NO: 38 - DNA, 143 bp, Artificial Sequence; identical to SEQ ID NO: 37
  Description of Artificial Sequence: Synthetic polynucleotide

SEQ ID NO: 39 - DNA, 208 bp, Artificial Sequence
  Description of Artificial Sequence: Synthetic polynucleotide
aggaacccctagtgatggagttggccactccctctctgggattgggattgcgcgctcgctcgcgggattgggattgggattgggattgggattgggattgataaaaatcaatcccaatcccaatcccaatcccaatcccaatcccgcgagcgagcgcgcaatcccaatcccagagagggagtggccaactccatcactaggggttcct

SEQ ID NO: 40 - DNA, 199 bp, Artificial Sequence
  Description of Artificial Sequence: Synthetic polynucleotide
aggaacccctagtgatggagttggccactccctctctgcgcgctcgctcgctcgggattgggattgggattgggattgggattgggattgataaaaatcaatcccaatcccaatcccaatcccaatcccaatcccgcgagcgagcgcgcaggagagggagtggccaactccatcactaggggttcctaagcttattata

SEQ ID NO: 41 - DNA, 154 bp, Artificial Sequence
  Description of Artificial Sequence: Synthetic polynucleotide
aggaacccctagtgatggagttggccactccctctctgcgcgctcgctcgctcactgagggcgcctataaagataaaaatccaggctttgcctgcctcagttagcgagcgagcgcgcagagagggagtggccaactccatcactaggggttcct

SEQ ID NO: 42 - DNA, 127 bp, Artificial Sequence
  Description of Artificial Sequence: Synthetic polynucleotide
ctagtgatggagttggccactccctctctgcgcgctcgctcgctcactgagggataaaaatccaggctttgcctgcctcagtgagcgagcgagcgcgcagagagggagtggccaactccatcactag